(19) United States
(12) Patent Application Publication
(10) Pub. No.: US 2011/0123512 A1
(43) Pub. Date: May 26, 2011

(54) METHOD OF REGULATING THE HEAT SHOCK RESPONSE

(76) Inventors: Veena Prahlad, Chicago, IL (US); Richard I. Morimoto, Evanston, IL (US)

(21) Appl. No.: 12/941,596

(22) Filed: Nov. 8, 2010

Related U.S. Application Data
(63) Continuation of application No. PCT/US09/43344, filed on May 8, 2009.
(60) Provisional application No. 61/051,593, filed on May 8, 2008.

(51) Int. Cl. A61K 31/7105; A61K 31/7076; A61K 31/397; A61K 38/06; A61K 31/4015; A61K 31/18; A61K 31/336; A61K 38/46; A61K 31/202; A61K 31/5575; A61K 31/19; A61K 31/352; A61K 38/02; A61P 25/00; A61P 25/28; A61P 11/00; A61K 38/18; A61P 25/16; A61P 43/00

(52) U.S. Cl. 424/94.6; 514/44 A; 514/46; 514/210.01; 514/21.9; 514/423; 514/604; 514/450; 514/183; 514/560; 514/573; 514/557; 514/457; 514/1.1; 514/17.7; 514/17.8; 514/1.8; 514/8.9

(57) ABSTRACT

The present invention is directed to method of modulating a heat shock response in a first cell of a multicellular organism comprising stimulating or inhibiting an HSR signaling activity of a second cell, wherein the second cell is a neuronal cell that regulates heat shock response activation in the first cell and that does not directly innervate the first cell.

### RELATED APPLICATIONS

This application is a continuation of International Application No. PCT/US09/43344, which designated the United States and was filed on May 8, 2009, published in English, which claims the benefit of U.S. Provisional Application No. 61/051,593, filed on May 8, 2008. The entire teachings of the above applications are incorporated herein by reference.

### GOVERNMENT SUPPORT

This invention was made with government support under Grant No. GM38109 awarded by the National Institutes of Health. The government has certain rights in the invention.

### BACKGROUND OF THE INVENTION

Cells normally maintain a balance between protein synthesis, folding, trafficking, aggregation, and degradation, referred to as protein homeostasis, utilizing sensors and networks of pathways [Sitia et al., Nature 426: 891-894, 2003; Ron et al., Nat Rev Mol Cell Biol 8: 519-529, 2007]. The cellular maintenance of protein homeostasis, or proteostasis, refers to controlling the conformation, binding interactions, location and concentration of individual proteins making up the proteome. Protein folding in vivo is accomplished through interactions between the folding polypeptide chain and macromolecular cellular components, including multiple classes of chaperones and folding enzymes, which minimize aggregation [Wiseman et al., Cell 131: 809-821, 2007]. Metabolic enzymes also influence cellular protein folding efficiency because the organic and inorganic solutes produced by a given compartment effect polypeptide chain salvation through non-covalent forces, including the hydrophobic effect, that influences the physical chemistry of folding. Metabolic pathways produce small molecule ligands that can bind to and stabilize the folded state of a specific protein, enhancing folding by shifting folding equilibria [Fan et al., Nature Med., 5, 112 (January 1999); Hammarstrom et al., Science 299, 713 (2003)]. Whether a given protein folds in a certain cell type depends on the distribution, concentration, and subcellular localization of chaperones, folding enzymes, metabolites and the like [Wiseman et al.]. Human loss of function diseases are often the result of a disruption of normal protein homeostasis, typically caused by a mutation in a given protein that compromises its cellular folding, leading to efficient degradation [Cohen et al., Nature 426: 905-909, 2003]. Human gain of function diseases are similarly frequently the result of a disruption in protein homeostasis leading protein aggregation [Balch et al. (2008), Science 319: 916-919].

At the cellular level, the heat shock response (HSR) protects cells against a range of acute and chronic stress conditions [Westerheide et al., J. Biol. Chem. 280(39): 33097 (2005)]. The heat shock response is a genetic response to environmental and physiological stressors resulting in a repression of normal cellular metabolism and a rapid induction of heat shock protein (HSP) genes expressing molecular chaperones, proteases and other proteins that are necessary for protection and recovery from cellular damage as a result of protein misfolding and aggregation [Westerheide et al.]. The heat shock response is mediated by the transcription factor, heat shock factor-1 (HSF-1). Although the HSPs protect cells against damage caused by various stressors, accumulation of large amounts of HSPs can be detrimental for cell growth and division [Morimoto et al. (1998), Genes Dev. 12, 3788]. Because HSP gene induction occurs at the cellular level and because isolated cells in tissue culture and individual cells within multicellular organisms produce a heat shock response when exposed to heat, the heat shock response has heretofore been considered to be cell-autonomous.

Both dysfunction in proteostasis and the heat shock response have been implicated in a diverse range of diseases including for example, cancer, neurodegenerative disease, metabolic diseases, inflammatory disease and cardiovascular disease. There remains a need in the art for therapeutic approaches to treat conditions associated with proteostasis dysfunction and/or altered induction of heat shock proteins.

### SUMMARY OF THE INVENTION

The present invention is based on the surprising discovery that the heat shock response in multicellular organisms is mediated by neuronal signaling. For example, Example 1 shows that the heat shock response in somatic cells of Caenorhabditis (C.) elegans is not cell-autonomous, but instead, depends on the thermosensory neurons, AFD, which regulate temperature-dependent behavior.

The present invention is directed to a method of modulating a heat shock response (HSR) in a first cell of a multicellular organism comprising stimulating or inhibiting the HSR signaling activity of a second cell. The second cell is a neuronal cell that regulates heat shock response activation in the first cell. In some embodiments, the second cell does not directly innervate the first cell. In another embodiment, the modulation of a heat shock response is mediated by inhibiting or stimulating the release of an HSR signaling factor from the second cell. In yet another embodiment, the modulation of a heat shock response is mediated by agonizing or antagonizing a receptor of the HSF signaling factor. In a further embodiment, the modulation of a heat shock response is mediated by agonizing or antagonizing a receptor on the first cell wherein the receptor mediates the effect of HSR signaling activity on the first cell.

In one embodiment, the HSF signaling factor is selected from the group consisting of a ligand of the IL/IGF signaling pathway, a ligand of the TGF-β signaling pathway, a ligand of the steroid hormone pathway and a neuropeptide.

In another embodiment, the invention is directed to a method of activating a heat shock response by stimulating the HSR signaling activity of the second cell. In yet another embodiment, the HSR signaling activity of the second cell is activated by stimulating the release of an HSR signaling factor from the second cell. In a further embodiment, the HSR signaling activity is increased by agonizing a receptor of the HSR signaling factor.

In an additional embodiment, the invention is directed to a method of suppressing a heat shock response in a first cell by inhibiting the HSR signaling activity of the second cell. In another embodiment, the HSR signaling activity of the second cell is suppressed by inhibiting the release of an HSR signaling factor from the second cell. In a further embodiment, the HSR signaling activity is decreased by antagonizing a receptor of the HSR signaling factor.

In an additional embodiment, the heat shock response is modulated by the administration of a pharmacologic agent in an amount sufficient to modulate HSR signaling activity. In one embodiment, the pharmacologic agent increases the HSR signaling activity of a neuronal cell, wherein the neuronal cell mediates HSR activation in a first cell. In another embodiment, the pharmacologic agent is administered in an amount sufficient to stimulate the release of an HSR signaling factor from the neuronal cell. In a further embodiment, the pharmacologic agent agonizes a receptor of the HSR signaling factor.

In yet another embodiment, a pharmacologic agent inhibits the HSR signaling activity of a neuronal cell, wherein the HSR signaling activity of the neuronal cell mediates HSR activation in a first cell. In a further embodiment, the neuronal cell does not directly innervate the first cell. In an additional embodiment, the pharmacologic agent is administered in an amount sufficient to inhibit the release of an HSR signaling factor. In another embodiment, the pharmacologic agent antagonizes a receptor of the HSR signaling factor. In yet another embodiment, the HSR signaling activity of the neuronal cell is suppressed by RNA or DNA interference.

In certain aspects, the invention is directed to a method of stimulating a heat shock response in a first cell comprising agonizing a receptor on the first cell, wherein the receptor mediates the effect of an HSR signaling factor on the first cell. In a further embodiment, the invention is directed to a method of suppressing a heat shock response in a first cell comprising antagonizing a receptor on the first cell, wherein the receptor mediates the effect of an HSR signaling factor on a receptor the first cell.

In another embodiment, the invention is a method of treating a patient suffering from a condition associated with a dysfunction in the homeostasis of a protein in a first cell of the patient comprising stimulating the HSR signaling activity of a second cell, wherein the second cell is a neuronal cell that regulates heat shock response activation in the first cell. In some embodiments, the second cell does not directly innervate the first cell. In one embodiment, the condition associated with a dysfunction in protein homeostasis is a loss of function disorder. In an additional embodiment, the condition associated with a dysfunction of protein homeostasis is a gain of function disorder.

In yet another embodiment, the invention is a method of treating a patient suffering from a condition associated with increased expression of a heat shock protein in a first cell in a patient comprising inhibiting the HSR signaling activity of a second cell, wherein the second cell is a neuronal cell that regulates heat shock response activation in the first cell. In another embodiment, the second cell does not directly innervate the first cell. In one embodiment, the condition associated with increased expression of a heat shock protein is cancer or a tumor. In a further embodiment, the condition associated with increased expression of a heat shock protein is a viral infection.

In a further embodiment, the invention is a method of modulating the HSR in a multicellular organism comprising stimulating or inhibiting TGF-β signaling activity. In another embodiment, the invention is directed to a method of modulating a heat shock response (HSR) in a first cell of a multicellular organism comprising stimulating or inhibiting the TGF-β signaling activity of a second cell, wherein the second cell is a neuronal cell. In some embodiments, the second cell does not directly innervate the first cell. In one embodiment, the heat shock response is activated by stimulating TGF-β signaling activity. In another embodiment, the heat shock response is inhibited by inhibiting TGF-β signaling activity.

In an additional embodiment, the invention is a composition comprising an isolated HSR signaling factor. In a further embodiment, the composition is a pharmaceutical composition comprising a pharmaceutically acceptable carrier and an isolated HSR signaling factor.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A is a schematic depicting genes affecting AFD and AIY functions.

FIG. 1B is a bar graph showing total hsp70 (C12C8.1) mRNA quantities in gcy-8 and ttx-3 mutants compared to wild-type animals before 30° C. heat shock (H.S.) and 2 hours (h) post heat shock.

FIG. 1C is a bar graph showing total hsp70 (C12C8.1) mRNA quantities in gcy-8 and ttx-3 mutants compared to wild-type animals before 34° C. heat shock (H.S.) and 2 hours (h) post heat shock.

FIG. 1D is a plot of the time course of hsp70 (C12C8.1) mRNA accumulation after 34° C. heat shock for 15 minutes (min) in gcy-8 and ttx-3 mutants compared to wild-type animals.

FIG. 1E is a plot of the time course of hsp70 (F44E5.4) mRNA accumulation after 34° C. heat shock for 15 minutes (min) in gcy-8 and ttx-3 mutants compared to wild-type animals.

FIG. 1F is a plot of the time course of hsp16.2 mRNA accumulation after 34° C. heat shock for 15 minutes (min) in gcy-8 and ttx-3 mutants compared to wild-type animals.

FIG. 1G is a bar graph showing the percentage of wild-type, gcy-8, ttx-3 and hsf-1 mutant animals

FIGS. 1H, 1I and 1J are photographs showing hsp70 (C128C.1) promoter-GFP reporter expression in wild-type, gcy-8 and ttx-3 mutants, respectively, 2 h after 34° C. heat shock for 15 mins. (i) indicates the pharynx, (ii) indicates the spermatheca, and (iii) indicates the intestinal cell.

FIG. 2A is a bar graph showing total hsp70 (C12C8.1) mRNA wild-type and gcy-8 mutant animals subjected to RNAi mediated knockdown of hsf-1 or daf-16 2 h after heat shock at 34° C. for 15 minutes.

FIG. 2B is a bar graph showing total hsp70 (C12C8.1) and cdr-1 mRNA quantities in wild-type and gcy-8 mutants after cadmium stress.

FIG. 2C is a bar graph showing total hsp70 (C12C8.1) mRNA quantities in cadmium-treated wild-type animals and gcy-8 mutants subjected to RNAi-mediated knockdown of hsf-1.

FIG. 3A is a bar graph showing total hsp70 (C12C8.1) mRNA quantities before and 2 h after 34° C. heat shock for 15 min in wild-type and gcy-8 mutant animals grown at low population densities or exposed to dauer pheromone 10 min before and during the 2 h of recovery after heat shock.

FIG. 3B is model depicting the regulation of cellular heat shock response by AFD-dependent signalling of temperature and dauer pheromone dependent signalling of growth conditions.

FIGS. 4A-4B are plots showing: (A) The rate of temperature increase, averaged across 10 random, well spaced points on a 6 mm thick agarose plate used for the heat shock experiments when plates were transferred from 20° C. to a 30° C. water bath for 15 minutes, and (B) when plates were transferred to a 34° C. water bath for 15 minutes. Both the wild-type and thermosensory mutant animals are exposed to the same temperature during heat shock.

FIGS. 4C-4F are photographs of a 0.008″ thick thermochromic Liquid Crystal (LC) ink plastic film which changes colour to indicate temperature (red=30° C. and blue=34° C.) showing: (C) A photograph of a 0.008″ thick thermochromic Liquid Crystal (LC) ink plastic film which changes colour to indicate temperature (red=30° C. and blue=34° C.) when applied to the surface of an agarose plate at 20° C. (D) The LC film after applied to the surface of an agarose plate immersed uniformly in a 30° C. water bath for 15 minutes. (E) The LC film after being applied to the surface of an agarose plate immersed uniformly in a 34° C. water bath for 15 minutes. (F) The LC sheet when applied to the surface of an agarose plate exposed to a temperature gradient of 25-34° C. for 15 minutes by immersing half of the plate in the 34° C. water bath, while the other half remained at room temperature of 25° C.

FIG. 4G is a bar graph showing the surface area of 30 images each of wild-type, gcy-8 and ttx-3 thermosensory mutant animals.

FIGS. 5A-C are photographs of hsp70 (C12C8.1) promoter-GFP reporter expression in (A) wild-type, (B) gcy-8, and (C) ttx-3 mutant animals 24 hours post-heat shock (34° C.; 15 minutes).

FIGS. 6A-B are bar graphs showing: A) Basal hsf-1 mRNA levels in wild-type and gcy-8 and ttx-3 mutants. (B) Basal mRNA levels of daf-16, hsp90 (daf-21) and hsp70 (hsp-1), in wild-type and gcy-8 mutants. mRNA levels were measured relative to the wild-type strain, by quantitative RT-PCR.

FIGS. 7A-7C are photographs showing the requirement of active neuronal signaling for heat shock gene expression; hsp70 (C12C8.1) promoter-GFP reporter expression assayed 2 hours post-heat shock in (A) control, non-anesthetized wild-type worms, (B) wild-type worms anesthetized with VA during heat shock, and (C) wild-type worms anesthetized with VA following heat shock.

FIG. 7D is a bar graph showing total hsp70 (C12C8.1) mRNA levels 2 hours post-heat shock in control non-anesthetized worms, and worms anesthetized with VAs (halothane and isoflurane) (pair-wise t-test; P value=0.001 and 0.0001 respectively).

FIG. 7E is a plot showing the percentage of wild-type or gcy-8 mutant animals expressing the hsp70 (C12C8.1) promoter-GFP reporter at various times post-heat shock. Heat shock in all experiments was 34° C. for 15 minutes. mRNA levels were measured by quantitative RT-PCR and normalized to wild-type values.

FIG. 8A-8C are photographs showing that the knock-down of dbl-1 mRNA inhibits the heat shock dependent expression of hsp70p (C12C8.1)::GFP. (A) Control animals express hsp70p (C12C8.1)::GFP in numerous tissue (spermatheca, pharynx and intestine) following heat shock; (B) Knock down of gcy-8 mRNA serves as a positive control for the RNAi screen, and inhibits heat shock induction of hsp70p (C12C8.1)::GFP; (C) dbl-1 RNAi inhibits heat shock induction of hsp70p (C12C8.1)::GFP.

FIG. 9 is a bar graph of hsp70 (C12C8.1) mRNA levels showing that knock-down of genes in the DBL-1 signaling pathway results in an aberrant heat shock-dependent expression of hsp70 (C12C8.1) mRNA throughout the animal.

FIG. 10 is a bar graph showing hsp70 (C12C8.1) mRNA levels showing that mutant dbl-1 loss of function animals have a deficient heat shock response, and express lower amounts of hsp70 (C12C8.1) mRNA. Animals overexpressing dbl-1 in their neurons have an accentuated expression of hsp70 (C12C8.1) mRNA.

FIGS. 11A and 11B are bar graphs of mRNA levels showing: (A) Animals carrying mutations in osm-9 and ocr-2 have defective chemosensory neurons and are deficient in their ability to upregulate cdr-1 and mtl-1, two genes products that are induced in the intestine upon exposure to cadmium. (B) osm-9 and ocr-2 animals express hsp70 (C12C8.1) mRNA following heat shock.

FIG. 12 is a drawing depicting the design of primers used to genotype the rrf-3deletion.

### DETAILED DESCRIPTION OF THE INVENTION

A description of preferred embodiments of the invention follows.

As used herein, the words “a” and “an” are meant to include one or more unless otherwise specified. For example, the term “a cell” encompasses both a single cell and a combination of two or more cells.

The present invention is based on the discovery that neuroendocrine signaling integrates behavioral, metabolic and stress-related responses to establish an organismal response to environmental or physiologic change. Because the cells involved in this neuronal signaling do not directly innervate the downstream cells or tissues in which heat shock gene induction is affected, it has been discovered that these signaling pathways are mediated by an HSF signaling factor released from the neuronal cells. The heat shock response has been implicated in a diverse set of diseases, including, for example, cancer, neurodegenerative diseases, lysosomal storage diseases, cardiovascular diseases and metabolic diseases. The present invention is directed to a method of modulating the neuronal signaling that mediates the induction of the heat shock response in downstream cells and tissues.

In certain embodiments, the present invention is a method of modulating a heat shock response (HSR) in a first cell of a multicellular organism comprising stimulating or inhibiting the HSR signaling activity of a second cell, wherein the second cell is a neuronal cell that regulates heat shock response activation in the first cell. In one embodiment, the second cell does not directly innervate the first cell.

The terms “HSR signaling activity” and “activity” in reference to the “second cell” or a neuronal cell refer to the neuroendocrine signaling of the neuronal cell that mediates a heat shock response in the first cell. The HSR signaling activity of a neuronal cell can be modulated by any means that results in a change in heat shock activation in the first cell. The HSR signaling activity of a neuronal cell is mediated by the release of an HSR signaling factor. The terms “HSR signaling activity” and “activity of the HSR signaling factor” are used interchangeably herein. One method of modulating the HSR signaling activity of a second cell is by inhibiting or stimulating the effect of an HSR signaling factor released by a neuronal cell. The effect of an HSR signaling factor can be increased or decreased by inhibiting or stimulating the release of the HSR signaling factor from the second cell. The effect or release of an HSR signaling factor is inhibited when there is a net decrease in the effect or release of the HSR signaling factor. The effect or release of an HSR signaling factor is stimulated when there is a net increase in the effect or release of the HSR signaling factor. Another method of modulating the HSR signaling activity of a second cell is by agonizing or antagonizing a receptor of the HSR signaling factor. The HSR signaling activity of a neuronal cell (or the “second cell”) is inhibited or stimulated when there is a net decrease or increase, respectively, in the activity of the neuronal cell that mediates a heat shock response in the first cell.

An “HSR signaling factor” is a chemical entity that in a multicellular organism is released from a neuronal cell and that directly or indirectly mediates the activation of a heat shock response in another cell. The term “chemical entity” is meant to encompass proteins, peptides, small molecules and the like. An “isolated HSR signaling factor” is a chemical entity that directly or indirectly mediates the activation of a heat shock response in a cell and that is in an isolated form. The HSR signaling factor can be isolated from a multicellular organism, produced recombinantly, produced by tissue culture or synthesized chemically. In one embodiment, the HSR signaling factor is a peptide. As used herein, the term “peptide” encompasses peptides having two or more amino acids. The term “peptide” explicitly includes proteins. A “receptor of an HSR signaling factor” or “HSR signaling factor receptor” is a receptor on a cell that binds to the HSR signaling factor and mediates the effects of the HSR signaling factor.

The HSR signaling factor can be a ligand of the IL/IGF signaling pathway, a ligand of the TGF-β signaling pathway, a ligand of the steroid hormone pathway or a neuropeptide or neuropeptide-like molecule. The HSR signaling factor receptor is a receptor of the IL/IGF receptor family, a receptor of the TGF-β superfamily, a receptor of the steroid receptor family or a receptor of a neuropeptide.

In one embodiment, the HSR signaling factor is a ligand of the IL/IGF signaling pathway. A ligand of the IL/IGF signaling pathway is a ligand of a receptor belonging to the IL/IGF receptor family. The IL/IGF receptor family has been described in Blakesley et al. (1996), Cytokine Growth Factor Res. 7(2): 153-9 and Werner et al. (2008), Arch Physiol Biochem 114(1): 17-22, the contents of each of which are incorporated by reference herein. The IL/IGF receptor family includes IGF receptors, insulin receptors and insulin-related receptors (Dissen et al. (2006). Endocrin. 147(1): 155-165). The IL/IGF receptors are related members of the tyrosine-kinase receptor superfamily of growth factor receptors. Both IGF-1R and IR are comprised of two α and two β subunits. These receptors have an extracellular ligand binding domain, a single transmembrane domain, and a cytoplasmic domain displaying the tyrosine kinase activity. As described below, the ligands of the IL/IGF signaling pathway that reduced heat shock induction of hsp70p:GFP throughout C. elegans, when gene expression is knocked down, are insulin-2 (ins-2), insulin-18 (ins-18) and insulin-23 (ins-23). In one embodiment, the ligand of the IL/IGF signaling pathway is ins-2, ins-18 or ins-23 or a mammalian homolog thereof.

In another embodiment, the HSR signaling factor belongs to the TGF-β superfamily. In one embodiment, the HSR signaling factor is a ligand of the TGF-β superfamily. In another embodiment, the HSR signaling factor receptor is a receptor of the TGF-β superfamily. A ligand of the TGF-β signaling pathway is a ligand of a receptor belonging to the TGF-β receptor superfamily. The superfamily can be divided into two general types: the bone morphogenetic factors (BMP)/growth/differentiation factors and the TGF-β member proteins. The TGF-β superfamily includes, but is not limited to, the five forms of TGF-β (TGF-β1-β5), differentiation factors (e.g., Vg-1), the hormones activin and inhibin, the Mulleriani inhibiting substance (MIS), osteogenic and morphogenic proteins (e.g., OP-1, OP-2, OP-3, and other BMPs), the developmentally regulated protein Vgr-1, and the growth/differentiation factors (e.g., GDF-1, GDF-3, GDF-9 and dorsalin-1) (US Pat. Pub. No. 20090042780). Morphogenic proteins of the TGF-β superfamily include, for example, the mammalian osteogenic protein-1 (OP-1, also known as BMP-7), osteogenic protein-2 (OP-2, also known as BMP-8), osteogenic protein-3 (OP3), BMP-2 (also known as BMP-2A or CBMP-2A, and the Drosophila homolog DPP), BMP-3, BMP-4 (also known as BMP-2B or CBMP-2B) and its C. Elegans homolog DBL-1, BMP-5, BMP-6 and its murine homolog Vgr-1, BMP-9, BMP-10, BMP-11, BMP-12, GDF3 (also known as Vgr2), GDF-8, GDF-9, GDF-10, GDF-11, GDF-12, BMP-13, BMP-14, BMP-15, GDF-5 (also known as CDMP-1 or MP52), GDF-6 (also known as CDMP-2 or BMP13), GDF-7 (also known as CDMP-3 or BMP-12). In another embodiment, the HSR signaling factor binds to a TGF-β receptor. As described below, the ligands or receptors of the TGF-β signaling pathway that reduce heat shock induction of hsp70p:GFP throughout C. elegans, when gene expression is knocked down, are dbl-1, daf-4, sma-9, tig-2, unc-129 and lin-31. In one embodiment, the HSR signaling factor is dbl-1, daf-4, sma-9, tig-2, unc-129 and lin-31, or a mammalian homolog of any of thereof. In another embodiment, the HSR signaling factor is a mammalian homolog of dbl-1.

A ligand of the steroid hormone pathway is a ligand of a steroid hormone receptor. Steroid hormone receptor proteins are members of the nuclear receptor family of proteins and are inducible transcription factors that transduce the effects of hormones into gene transcription. These receptors are activated by the binding of a ligand which induces the receptors to bind to specific response elements in the promoter regions of target genes, hormone responsive elements, inducing transcription of certain genes. Steroid receptors include, for example, estrogen, progesterone, androgen, Vitamin D, cis-retonic acid, Nurr-1, thyroid hormone, mineralocorticoids and glucocorticoid. As described below, the ligands of the steroid hormone signaling pathway that, when knocked down, reduced heat shock induction of hsp70p:GFP throughout C. elegans are daf-9 and daf-12. In one embodiment, the ligand of the steroid hormone signaling pathway is daf-9, daf-12, or a mammalian homolog of any of thereof.

In yet another embodiment, the HSR signaling factor is a neuropeptide or neuropeptide-like molecule. Neuropeptides are short peptides that mediate synaptic activity or that function as primary neurotransmitters (Li et al. (2008). Neuropeptides. http://www.wormbook.org/chapters/www neuropeptides/neuropeptides.html, the contents of which are incorporated by reference herein). The majority of neuropeptides can be divided into two families: insulin-like peptides and FMRF amide (Phe-Met-Arg-Phe-NH₂)-related peptides (referred to as FLPs in C. elegans). Non-insulin, non-FLP peptides in C. elegans are referred to as neuropeptide-like proteins or neuropeptide-like molecules. As used herein, neuropeptide-like molecules are expressly included by use of the term “neuropeptide” unless otherwise indicated. As described below, the neuropeptide-like molecules that, when knocked down, reduce heat shock induction of hsp70p:GFP throughout C. elegans are neuropeptide like proteins (nlp)-4, nlp-5, nlp-7, nlp-21 and nlp-22. In one embodiment, the neuropeptide or neuropeptide-like molecule is nlp-4, nlp-5, nlp-7, nlp-21, nlp-22 or a mammalian homolog of any of thereof.

Homologs are defined herein as sequences characterized by nucleotide or amino acid sequence homology. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md.

In certain embodiments, the HSR signaling factor mediates heat shock activation by directly or indirectly regulating the expression of one or more transcription factors in the cell in which the heat shock response is activated (referred to herein as the “first cell”). In other embodiments, the HSR signaling factor increases the expression of one or more transcription factors. Transcription of several heat shock protein genes is controlled by heat shock factors [Shoenx et al. (2001), Physiol. Rev. 81: 1461-1497]. The heat shock transcription factors, HSF1, HSF2, and HSF4 have been identified in humans [Shoenx et al.]. In one embodiment, the transcription factor is HSF-1.

The “first cell” or the cell in which the heat shock response is activated can be any single cell or group of cells. In some embodiment, the first cell is not directly innervated by a neuronal cell that releases the HSR signaling factor. In other embodiments, the first cell is directly innervated by the neuronal cell that releases the HSR signaling factor. Exemplary cells include neuronal cells, muscle cells (e.g., skeletal muscle cells, and cardiac muscle cells such as pacemaker cells, atrial cells, atrial-ventricular nodal cells, left ventricular cells, right ventricular cells, papillary muscle cells, and Purkinje fiber cells and smooth muscle cells), blood cells, kidney cells, epithelial cells, intestinal cells, lymph node cells, spleen cells, hepatic cells, thymic cells, salivary gland cells, pituitary cells, bladder cells, bone cells, breast cells, cervical cells, colorectal cells, kidney cells, laryngeal cells, pulmonary cells, lymphatic cells, skin cells and haematopoietic cells (such as for instance T lymphocytes, B lymphocytes, macrophages, dendritic cells and progenitors thereof). In one embodiment, the cell in which the heat shock response is activated is a non-neuronal cell. In another embodiment, the heat shock response is activated in a group of cells. In yet another embodiment, the heat shock response is activated in a group of cells in a tissue. In a further embodiment, the heat shock response is activated in a group of cells in an organ.

The “second cell” or the neuronal cell that mediates the induction of heat shock response in a first cell or possesses HSR signaling activity or from which the HSR signaling factor is released can be any single neuronal cell or group of neuronal cells. In some embodiments, the second cell t does not directly innervate the cell in which a heat shock response is activated. In another embodiment, the second cell directly innervates the cell in which the heat shock response is activated. In one embodiment, the neuronal cell is a thermosensory neuronal cell. In vertebrates, thermosensory neurons are located in the trigeminal and dorsal root ganglia [Bandell et al. (2007). Curr. Opin. Neurobiol., 17(4):490-497; Jordt et al. (2003), Curr. Opin. Neurobiol., 13:487-492; the contents of these references are hereby incorporated by reference]. Different groups of neurons respond to different temperature cues [Bandell et al. (2007)].

Heat shock was first discovered as a trigger of the heat shock response leading to enhanced transcription of certain genes [Snoeckx et al.]. The products of this transcriptional activity are called heat shock proteins [Snoeckx et al.]. Most heat shock proteins (Hsps) are named with reference to a molecular mass indication, for example, Hsp27. The classification of various Hsps in families is based on their related function and size. The size of heat shock proteins range from 10 to 170 kDa. Family names are conventionally written in capitals. For example, “HSP70” refers to the HSP70 family. The HSP70 family range in weight between 70 and 78 kDa. One example of an HSP70 family member is Hsp72 (commonly referred to as Hsp70). The heat shock response encompasses the induction of a gene encoding heat shock proteins. Heat shock protein genes that can be induced according to methods of the present invention include, but are not limited to, a gene encoding a protein from a family selected from the HSP10 family, the HSP40 family, the HSP60 family, the HSP70 family, the HSP90 family, the HSP100 family, the HSP27 family, the αA-crystallin family and the αB-crystallin family of proteins.

In some embodiments of the invention, the multicellular organism is an animal. Animals include vertebrates and invertebrates, e.g., mammals and non-mammals, including, but not limited to, sheep, dogs, cows, chickens, C. elegans, Drosophila melanogaster, amphibians, reptiles and humans. In one embodiment, the animal is an invertebrate. In another embodiment, the animal is a vertebrate. In a further embodiment, the animal is a mammal. In certain embodiments, the mammal is a human.

In certain aspects of the invention, the HSR signaling activity of a neuronal cell is stimulated. In one embodiment, a pharmacologic agent is administered to stimulate the HSR signaling activity of the neuronal cell. An amount of a pharmacologic agent sufficient to stimulate an HSR signaling activity in a cell is an amount that increases the HSR signaling activity relative to that in the cell or a cell of the same type in the absence of pharmacologic agent administration. One method of stimulating the HSR signaling activity of a neuronal cell (or the second cell of the present invention) is to stimulate the release of an HSR signaling factor from the neuronal cell and/or to agonize a receptor of an HSR signaling factor. The release of an HSR signaling factor can be stimulated by any means that increases the release of the factor from a neuronal cell. In one embodiment, a pharmacologic agent is administered to stimulate the release of an HSR signaling factor. An amount of a pharmacologic agent sufficient to stimulate the release of an HSR signaling factor is an amount that increases the release of the HSR signaling factor from a cell relative to that in the cell or same cell type in the absence of pharmacologic agent administration. In another embodiment, the pharmacologic agent is an agonist of a receptor of the HSR signaling factor. A pharmacologic agent that increases HSR signaling activity or HSR signal factor release or agonizes an HSR signaling factor receptor can be identified by measuring HSR signaling activity, HSR signaling factor receptor activity or HSR signaling factor release from the second cell after administration of a pharmacologic agent and comparing that with the HSR signaling activity or HSR signaling factor release in the absence of pharmacologic agent.

In certain other aspects of the invention, the HSR signaling activity of a neuronal or second cell is inhibited. In one embodiment, a pharmacologic agent is administered to inhibit the HSR signaling activity. An amount of a pharmacologic agent sufficient to inhibit an HSR signaling activity in a cell is an amount that decreases the HSR signaling activity relative to that in the cell or cell of the same type in the absence of pharmacologic agent administration. One method of inhibiting the HSR signaling activity of a neuronal cell is to inhibit the release of an HSR signaling factor from the neuronal cell. Another method of inhibiting the HSR signaling activity of a neuronal cell is to antagonize a receptor of an HSR signaling factor. The release of an HSR signaling factor can be inhibited by any means that decreases the release of the factor from a neuronal cell. In one embodiment, a pharmacologic agent is administered to inhibit the release of an HSR signaling factor. An amount of a pharmacologic agent sufficient to inhibit the release of an HSR signaling factor is an amount that decreases the release of the HSR signaling factor from a cell relative to the release of the HSR signaling factor in the cell or same cell type in the absence of pharmacologic agent administration. In another embodiment, the pharmacologic agent is an antagonist of a receptor of an HSR signaling factor. A pharmacologic agent that decreases HSR signaling activity or HSR signaling factor release or inhibits a receptor of the HSR signaling factor can be identified by measuring HSR signaling activity or HSR signaling factor release after administration of the pharmacologic agent and comparing that with HSR signaling activity or HSR signaling factor release in the absence of pharmacologic agent.

In one embodiment, the activity of an HSR signaling factor or receptor of an HSR signaling factor is inhibited using RNA or DNA interference. RNA and DNA interference encompass the use of short interfering RNAs (siRNA), short hairpin RNAs (shRNA), antisense RNA transcripts, antisense oligonucleotides and ribozymes. RNA interference is a mechanism of post-transcriptional gene silencing mediated by double-stranded RNA (dsRNA), [Jain, Pharmacogenomics 5: 239-42, 2004]. RNA interference is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. An siRNA can comprise two RNA strands hybridized together, or can alternatively comprise a single RNA strand that includes a self-hybridizing portion. A further method of RNA interference is the use of short hairpin RNAs (shRNA). Antisense RNA transcripts have a base sequence complementary to part or all of any other RNA transcript in the same cell. Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Certain nucleic acid molecules referred to as ribozymes or deoxyribozymes have also been shown to catalyze the sequence-specific cleavage of RNA molecules. The cleavage site is determined by complementary pairing of nucleotides in the RNA or DNA enzyme with nucleotides in the target RNA. Thus, RNA and DNA enzymes can be designed to cleave any RNA molecule, thereby increasing its rate of degradation [Cotten et al, EMBO J. 8: 3861-3866, 1989; Usman et al., Nucl. Acids Mol. Biol. 10: 243, 1996; Usman, et al., Curr. Opin. Struct. Biol. 1: 527, 1996; Sun, et al., Pharmacol. Rev., 52: 325, 2000. See also e.g., Cotten et al, EMBO J. 8: 3861-3866, 1989].

In other aspects, the invention is directed to a method of stimulating a heat shock response in a first cell comprising agonizing a receptor of an HSF signaling factor, or a receptor on the first cell, where the receptor on the first cell mediates the effect of an HSR signaling factor on the first cell. In a further embodiment, the invention is directed to a method of suppressing a heat shock response in a first cell comprising antagonizing a receptor of an HSF signaling factor, or a receptor on the first cell, wherein the receptor on the first cell mediates the effect of an HSR signaling factor on a receptor the first cell. A compound that binds to a receptor and mimics the effect of the natural ligand is an agonist or is said to agonize the receptor. In one embodiment, the receptor is agonized by the administration of an agonist. As used herein, agonists encompass full and partial agonists. A compound that inhibits the effect of the natural ligand is an antagonist or is said to antagonize the receptor. Receptor antagonists include competitive antagonists, non-competitive antagonists, uncompetitive antagonists and partial antagonists. In another embodiment, the HSF signaling factor receptor or receptor on the first cell is antagonized by the administration of an antagonist.

The invention also encompasses a method of treating a condition associated with a dysfunction in protein homeostasis in a patient in need thereof comprising stimulating the HSR signaling activity of a second cell, wherein the HSR signaling activity of the second cell mediates activation of the heat shock response of a first cell and wherein the second cell is a neuronal cell. In one embodiment, the second cell does not directly innervate the first cell. In one embodiment, the invention is a method of treating a condition associated with a dysfunction in protein homeostasis in a patient in need thereof comprising stimulating the release of a HSR signaling factor from the second cell. The HSR signaling activity of the second cell or the neuronal cell can be stimulated as described above by administering to the patient a pharmacologic agent that stimulates the HSR signaling activity of the second cell and/or stimulates the release of the HSR signaling factor from the second cell and/or agonizes a receptor of the HSR signaling factor.

“Treating” or “treatment” includes preventing or delaying the onset of the symptoms, complications, or biochemical indicia of a disease, alleviating or ameliorating the symptoms or arresting or inhibiting further development of the disease, condition, or disorder. A “patient” is a human subject in need of treatment.

The invention encompasses the treatment of a condition associated with a dysfunction in the homeostasis of a protein. Exemplary proteins include glucocerebrosidase, hexosamine A, cystic fibrosis transmembrane conductance regulator, aspartylglucsaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparin N-sulfatase, α-N-acetylglucosaminidase, α-glucosaminide N-acetyltransferase, N-acetylglucosamine-6-sulfate sulfatase, α-N-acetylgalactosaminidase, α-neuramidase, β-glucuronidase, β-hexosamine A and acid lipase, polyglutamine, α-synuclein, Ab peptide, tau protein and transthyretin.

In one embodiment, the disease associated with a dysfunction in proteostasis is a gain of function disorder. The terms “gain of function disorder,” “gain of function disease,” “gain of toxic function disorder” and “gain of toxic function disease” are used interchangeably. A gain of function disorder is a disease characterized by increased aggregation-associated proteotoxicity. In these diseases, aggregation exceeds clearance inside and/or outside of the cell. Gain of function diseases include, but are not limited to neurodegenerative diseases associated with aggregation of polyglutamine, Lewy body diseases, amyotrophic lateral sclerosis, transthyretin-associated aggregation diseases, Alzheimer's disease and prion diseases. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: extracellular aggregates of Aβ peptide and intracellular aggregates of the microtubule associated protein tau. Transthyretin-associated aggregation diseases include, for example, senile systemic amyloidoses and familial amyloidotic neuropathy. Lewy body diseases are characterized by an aggregation of α-synuclein protein and include, for example, Parkinson's disease. Prion diseases (also known as transmissible spongiform encephalopathies or TSEs) are characterized by aggregation of prion proteins. Exemplary human prion diseases are Creutzfeldt-Jakob Disease (CJD), Variant Creutzfeldt-Jakob Disease (vCJD), Gerstmann-Straussler-Scheinker Syndrome, Fatal Familial Insomnia and Kuru.

In yet another embodiment, the disease associated with a dysfunction in protein homeostasis is a loss of function disorder. The terms “loss of function disease” and “loss of function disorder” are used interchangeably. Loss of function diseases are a group of diseases characterized by inefficient folding of a protein resulting in excessive degradation of the protein. Loss of function diseases include, for example, cystic fibrosis and lysosomal storage diseases. In cystic fibrosis, the mutated or defective enzyme is the cystic fibrosis transmembrane conductance regulator (CFTR). One of the most common mutations of this protein is ΔF508 which is a deletion (Δ) of three nucleotides resulting in a loss of the amino acid phenylalanine (F) at the 508th (508) position on the protein. Lysosomal storage disease are a group of diseases characterized by a specific lysosomal enzyme deficiency which may occur in a variety of tissues, resulting in the build up of molecules normally degraded by the deficient enzyme. The lysosomal enzyme deficiency can be in a lysosomal hydrolase or a protein involved in the lysosomal trafficking Lysosomal storage diseases include, but are not limited to, aspartylglucosaminuria, Fabry's disease, Batten disease, Cystinosis, Farber, Fucosidosis, Galactasidosialidosis, Gaucher's disease (including Types 1, 2 and 3), Gm1 gangliosidosis, Hunter's disease, Hurler-Scheie's disease, Krabbe's disease, a-Mannosidosis, B-Mannosidosis, Maroteaux-Lamy's disease, Metachromatic Leukodystrophy, Morquio A syndrome, Morquio B syndrome, Mucolipidosis II, Mucolipidosis III, Neimann-Pick Disease (including Types A, B and C), Pompe's disease, Sandhoff disease, Sanfilippo syndrome (including Types A, B, C and D), Schindler disease, Schindler-Kanzaki disease, Sialidosis, Sly syndrome, Tay-Sach's disease and Wolman disease.

In another embodiment, the disease associated with a dysfunction in proteostasis and/or heat shock proteins is a cardiovascular disease. Cardiovascular diseases include, but are not limited to, coronary artery disease, myocardial infarction, stroke, restenosis and arteriosclerosis. Conditions associated with a dysfunction of proteostasis also include ischemic conditions, such as, ischemia/reperfusion injury, myocardial ischemia, stable angina, unstable angina, stroke, ischemic heart disease and cerebral ischemia.

The present invention also encompasses a method of treating a patient suffering from a condition associated with a dysfunction in proteostasis by increasing HSR signaling activity of a neuronal cell in combination with the administration of a compound that increases HSF-1 activity. Compounds that increase HSF-1 activity include, but are not limited to, protein synthesis inhibitors, proteasome inhibitors, a serine protease inhibitors, Hsp90 inhibitors, inflammatory mediators and triterpenoids. Exemplary protein synthesis inhibitors are puromycin and azetidine. Exemplary proteasome inhibitors are MG132 and lactacystin. Exemplary protease inhibitors are DCIC, TPCK and TLCK. Exemplary Hsp90 inhibitors are radicocol, geldanamycin and 17-AAG. Exemplary triterpenoids are celastrol and derivatives or analogues of celastrol. Exemplary inflammatory mediators are cyclopentanone prostaglandins, arachidonate and phospholipase A₂.

The invention also encompasses a method of treating a patient suffering from a condition associated with a dysfunction in proteostasis comprising stimulating the HSR signaling activity of a second cell (or a neuronal cell) in combination with the administration of a pharmacologic chaperone. Pharmacologic chaperones or kinetic stabilizers refer to compounds that bind an existing steady state level of the folded mutant protein and chemically enhance the folding equilibrium by stabilizing the fold [Bouvier, Chem Biol. 14: 241-242, 2007; Fan et al., Nat Med 5: 112-115, 1999; Sawkar et al., Proc Natl Acad Sci USA 99:15428-15433, 2002; Johnson and Kelly, Accounts of Chemical Research 38: 911-921, 2005]. The pharmacologic chaperone is administered in amount that in combination with stimulation of the HSR signaling activity of a second cell is sufficient to treat a patient suffering from a condition associated with a dysfunction in proteostasis. Exemplary pharmacologic chaperones are described in U.S. Patent Publication No.'s 20080056994, 20080009516, 20070281975, 20050130972, 20050137223, 20050203019, 20060264467 and 20060287358.

In another embodiment, the invention is a method of treating a patient suffering from a condition associated with a dysfunction in proteostasis by increasing the HSR signaling activity in combination with the administration of a mechanistically distinct proteostasis regulator. The term “proteostasis regulator” refers to small molecules, siRNA and biologicals (including, for example, proteins that enhance cellular protein homeostasis). Proteostasis regulators have been described, for example, in Balch et al. (2008). Science 319 (5865) 916-919 and Mu et al. (2008). PLoS Biology 6(2) e26 doi:10.1371/journal.pbio.0060026, the contents of each of which are herein incorporated by reference. Proteostasis regulators encompass pharmacologic agents that stimulate the HSR signaling activity of the second cell and/or stimulate the release of a HSR signaling factor from the second cell. Proteostasis regulators function by manipulating signaling pathways, including, but not limited to, the heat shock response or the unfolded protein response, or both, resulting in transcription and translation of proteostasis network components. Proteostasis regulators can also regulate protein chaperones by upregulating transcription or translation of the protein chaperone, or inhibiting degradation of the protein chaperone. In addition, proteostasis regulators can upregulate an aggregation pathway or a disaggregase activity. In one aspect, the proteostasis regulator is distinct from a chaperone in that the proteostasis regulator can enhance the homeostasis of a mutated protein but does not bind the mutated protein. A mechanistically distinct proteostasis regulator is a proteostasis regulator that enhances cellular proteostasis by a mechanism other than by modulating the HSR signaling activity of the second cell (including affecting the release of an HSR signaling factor from the second cell). Exemplary proteostasis regulators are celastrol, MG-132 and L-type Ca2+ channel blocker.

In one aspect, the invention is a method of treating a condition associated with increased expression of heat shock proteins in a patient in need thereof comprising suppressing the HSR signaling activity of the second cell wherein the second cell is a neuronal cell. In one embodiment, the second cell does not directly innervate the first cell. In one embodiment, the method of suppressing the HSR signaling activity of the second cell comprises inhibiting the release of an HSR signaling factor from a second cell. In another embodiment, the method of suppressing HSR signaling activity of the second cell comprises administering an antagonist of a receptor of an HSF signaling factor. In one embodiment, the condition associated with increased expression of a heat shock protein is cancer or a tumor. In another embodiment, the condition associated with increased expression of a heat shock protein is a viral infection. The HSR signaling activity of the second cell and release of the HSR signaling factor can be inhibited by the administration of pharmacologic agent in an amount sufficient to inhibit the release of a HSR signaling factor from a neuronal cell or inhibit the activity of a receptor of an HSR signaling factor. The HSR signaling activity of the second cell can also be inhibited by RNA or DNA interference.

The condition associated with increased expression of heat shock proteins can be cancer or a tumor. Cancers that can be treated according to methods of the present invention include, but are not limited to, breast cancer, colon cancer, pancreatic cancer, prostate cancer, lung cancer, ovarian cancer, cervical cancer, multiple myeloma, basal cell carcinoma, neuroblastoma, hematologic cancer, rhabdomyosarcoma, liver cancer, skin cancer, leukemia, basal cell carcinoma, bladder cancer, endometrial cancer, glioma, lymphoma, and gastrointestinal cancer.

In another embodiment, the invention is a method of treating cancer or a tumor comprising inhibiting the HSR signaling activity in combination with the administration of a chemotherapeutic agent. Chemotherapeutic agents that can be utilized include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

In a further embodiment, the invention is a method of treating cancer or a tumor comprising inhibiting HSR signaling activity in combination with radiation therapy.

In another embodiment, the condition associated with increased expression of a heat shock protein is a viral infection. In a further embodiment, the viral infection is caused by a virus selected from a tumor virus and an RNA virus. Exemplary tumor viruses are the herpes viruses, the papiloma viruses, the polyoma viruses and HTLV-1 [McCance et al., Human

Tumor Viruses, 1998, American Society for Microbiology]. Herpes viruses include, but are not limited to, EBV (HHV-4), HHV-6 and HHV-8. Papilomaviruses include, but are not limited to, HPV-1, -2, -4, -5, -6, -8, -6, -11, -16, -18, -31, -33, -35, -45, -51, -52, -58 and -58.

RNA viruses include, for example, arenaviridae, bunyaviridae, calciviridae, coronaviridae, filoviridae, flaviridae, orthomyxoviridae, paramyxoviridae, picornaviridae, reoviridae, rhabdoviridae, retroviridae, or togaviridae. Exemplary RNA viruses include, but are not limited to, the human coronaviruses, such as the SARS-Associated Coronavirus, human toroviruses associated with enteric and respiratory diseases; the Norwalk virus. Yellow Fever virus, West Nile virus, Hepatitis C virus, Dengue fever virus, Polio virus, the common cold virus, hepatitis A virus, hepatitis E, rotavirus, Borna disease virus; Bunyaviradae, such as Hanta virus, California encephalitis virus, Japanese encephalitis virus, LaCrosse virus, Rift Valley fever virus, Bunyavirus, Arbovirus, Ebola virus and Marburg virus; Influenza virus type A, Influenza virus type B, Influenza virus type C, Mumps virus, Measles virus, Subacute sclerosing panencephalitis (SSPE) virus and Respiratory syncytial virus (RSV).

In a further embodiment, the invention is a method of treating a patient suffering from a viral infection comprising inhibiting HSR signaling activity in combination with the administration of an anti-viral drug.

The invention also includes compositions comprising an isolated HSR signaling factor. In one embodiment, the invention is a pharmaceutical composition comprising a pharmaceutically acceptable carrier or excipient and an isolated HSR signaling factor.

The preferred form of a pharmacologic agents or pharmaceutical compositions described herein depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSE™, agarose, cellulose, and the like), polymeric amino acids, amino acid copolymers, and lipid aggregates (such as oil droplets or liposomes).

For parenteral administration, pharmaceutical compositions or pharmacologic agents can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

The compositions can be prepared as injectable formulations, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The compositions and pharmacologic agents described herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. Topical application can result in transdermal or intradermal delivery. Transdermal delivery can be achieved using a skin patch or using transferosomes. [Paul et al., Eur. J. Immunol. 25: 3521-24, 1995; Cevc et al., Biochem. Biophys. Acta 1368: 201-15, 1998].

The invention is illustrated by the following examples which are meant to be limited in any way.

### EXEMPLIFICATION

### Example 1

### Regulation of the Cellular Heat Shock Response in Caenorhabditis elegans by Thermosensory Neurons

Temperature pervasively affects all cellular processes. In response to a rapid increase in temperature, all cells undergo a heat shock response, an ancient and highly conserved program of stress-inducible gene expression, to re-establish cellular homeostasis. In isolated cells, the heat shock response is initiated by the presence of misfolded proteins and therefore thought to be cell-autonomous. In contrast, we show that within the metazoan Caenorhabditis elegans, the heat shock response of somatic cells is not cell-autonomous, but rather depends on the thermosensory neuron, AFD, which senses ambient temperature and regulates temperature-dependent behavior. We propose a model whereby this loss of cell autonomy serves to integrate behavioral, metabolic, and stress-related responses to establish an organismal response to environmental change.

The heat shock response counteracts the detrimental effects of protein misfolding and aggregation that results from biochemical and environmental stresses, including increases in temperature (1, 2). This response, orchestrated by the ubiquitously expressed heat shock factor-1 (HSF-1), involves the rapid transcription of a specific set of genes encoding the cytoprotective heat shock proteins (HSPs) (1, 2). The stress-induced appearance of non-native proteins imbalances cellular homeostasis, and the resulting shift in chaperone requirements is thought to trigger the heat shock response (1, 2). Since all these events occur at the cellular level the heat shock response is thought to be cell-autonomous. Indeed, isolated cells in tissue culture, unicellular organisms (1, 2), and individual cells within a multicellular organism (3) can all produce a heat shock response when exposed directly to heat.

Although the heat shock response is essential for the survival of cells exposed to stress, the accumulation of large amounts of HSPs can be detrimental for cell growth and division (2, 4). Therefore, while cellular autonomy in initiating this response may be beneficial for unicellular organisms and isolated cells, the uncoordinated triggering of the heat shock response in individual cells within a multicellular organism could interfere with the complex interactions between differentiated cells and tissues.

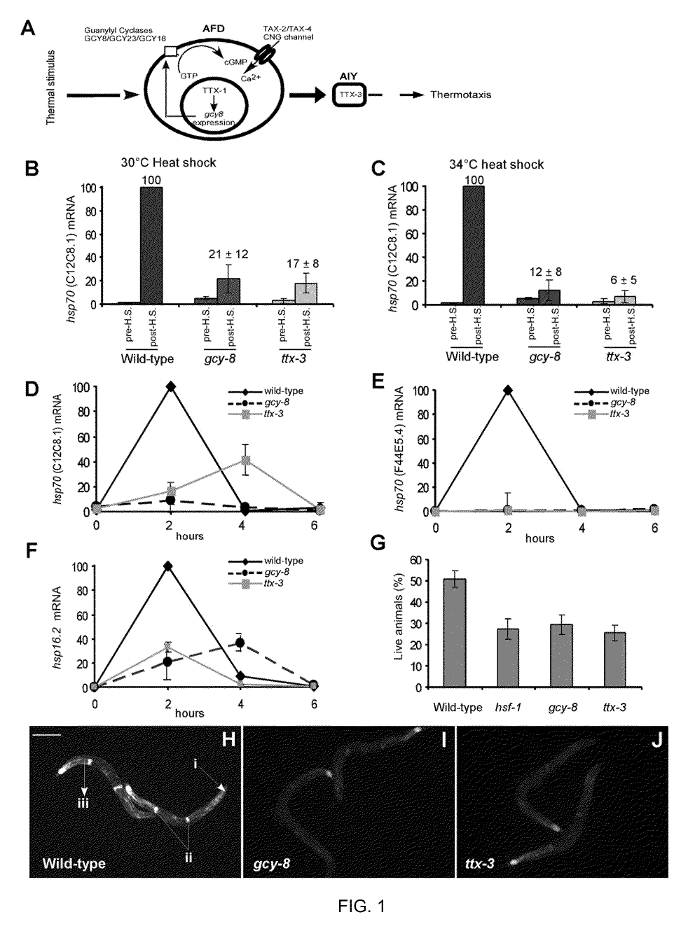

In C. elegans, a pair of thermosensory neurons, the AFDs detect and respond to ambient temperature (5, 6). The AFDs, and their postsynaptic partner cells, the AIYs, regulate the temperature-dependent behavior of the organism and are required for finding the optimal temperature for growth and reproduction (6). We tested whether this thermosensory neuronal circuitry also regulates the heat shock response of somatic cells. For this, we exposed wild-type C. elegans, or animals carrying loss of function mutations affecting the AFD or AIY neurons (FIG. 1A), to a transient increase in temperature and assayed their heat shock response. The mutations chosen [gcy-23, gcy-8 (7), tax-4, ttx-1 and ttx-3 (for details of these mutations see (8)] exclusively affect neuronal function as the wild-type gene products are not expressed in other tissues, with gcy-8 and gcy-23 expressed solely in the AFDs (7). Wild-type and mutant adult animals, grown at low population densities at 20° C. in the presence of abundant bacteria (8), were exposed to a transient increase in temperature (30° C. or 34° C. for 15 min.) and their heat-shock response was measured as the total amount of mRNA encoding the major heat-inducible cytoplasmic hsp70, C12C8.1(9), 2 hours after heat shock. Mutations affecting the AFD or AIY neurons reduced heat shock-dependent accumulation of hsp70 (C12C8.1) mRNA at both temperatures ((8), Table SI; FIGS. 1B and C), whereas a mutation, (ocr-2) (10), affecting four other sensory neurons of the animal, had no effect ((8) Table SI).

The decrease in hsp70 (C12C8.1) abundance was not merely due to a delay in the onset of heat shock response (FIG. 1D). The gcy-8 and ttx-3 mutants had consistently lower amounts of hsp70 (C12C8.1) mRNA compared to wild-type over a 6 hour period after heat shock, with some mRNA accumulation seen 4 hours post-heat shock. Mutants with defective AFD or AIY neurons also had reduced heat shock-dependent accumulation of another cytoplasmic hsp70, F44E5.4 (9), and the small heat shock protein, hsp16.2, mRNAs (11) (FIGS. 1E and F).

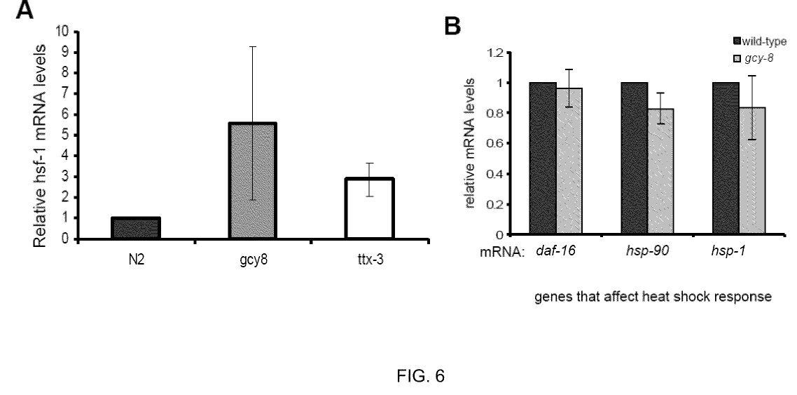

The diminished expression of HSP genes in the gcy-8 and ttx-3 mutants might make them less viable than wild-type animals under conditions of heat stress. This was the case (FIG. 1G): the decrease in thermotolerance of the thermosensory mutants was similar to that of animals carrying a hsf-1 loss-of-function allele (12) (FIG. 1G), although hsf-1 mRNA levels were not diminished in gcy-8 and ttx-3 mutants ((8), FIG. 6A).

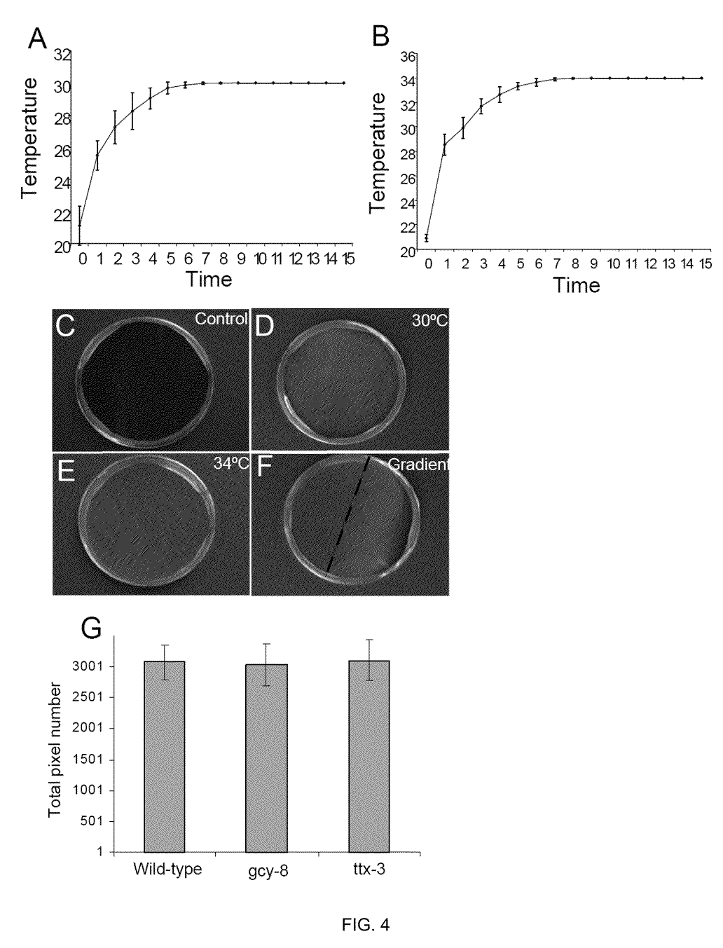

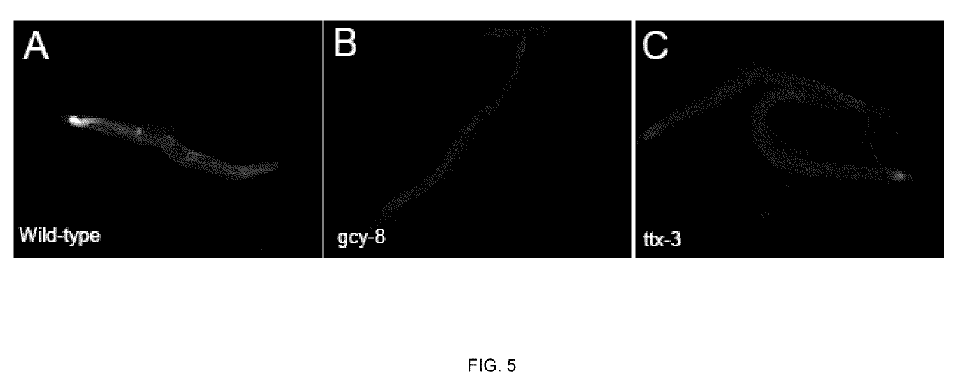

We examined whether the decreased accumulation of inducible hsp70 mRNA in the thermosensory mutants reflected selective reduction in neuronal tissue or corresponded to diminished expression in all cells throughout the animal. Heat shock promotes hsp mRNA expression in numerous somatic cells of wild-type C. elegans as monitored with a hsp70 (C12C8.1) promoter GFP reporter construct (13) (FIG. 1H). The expression of this hsp70 reporter in strains with mutant gcy-8 and ttx-3 genes was reduced in all somatic cells 2 hours after heat shock (FIGS. 1I and J) and continued to be impaired after 24 hours, although these somatic cells of the thermosensory mutants should have experienced the same heat-shock temperature as the equivalent wild-type cells ((8), FIGS. 4 and 5). These results indicate that the heat shock response in C. elegans is not cell-autonomous. Instead, the AFD and AIY neurons appear to regulate both the magnitude and the time course of heat shock gene expression in non-neuronal cells, influencing organismal thermotolerance. Subsequent experiments were done at 34° C. using the AFD-specific mutant, gcy-8 that is the most upstream component known in the thermosensory neuronal circuitry.

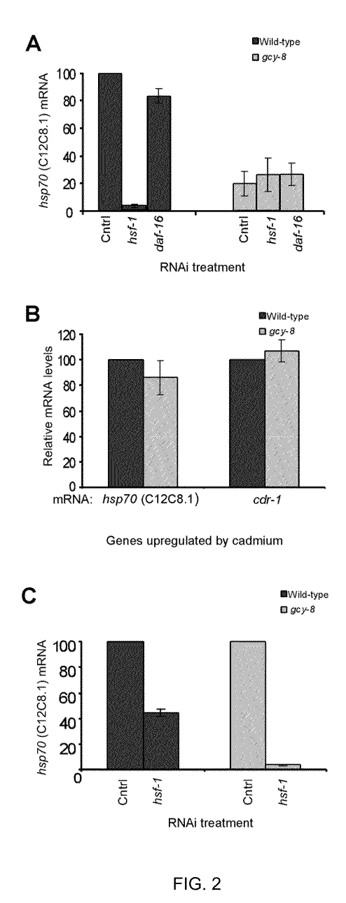

To control the heat shock response of non-neuronal cells, the AFD neurons must regulate the activities of cellular transcription factors. In eukaryotes, HSP expression after heat shock is HSF-1-dependent (2). Organismal thermotolerance also requires the FOXO transcription factor, DAF-16 (14). To test which transcription factor is required for AFD-dependent hsp70 (C12C8.1) mRNA expression following heat shock, we used RNA interference (RNAi). Depletion of hsf-1 mRNA, but not daf-16 mRNA in wild-type C. elegans decreased hsp70 (C12C8.1) expression throughout the organism (FIG. 2A). Depletion of hsf-1 mRNA, however, had no effect on the already diminished amounts of hsp70 (C12C8.1) mRNA in the gcy-8 mutants (FIG. 2A). Thus, AFD appears to regulate HSP expression through HSF-1, although we cannot rule out the existence of a parallel transcriptional mechanism.

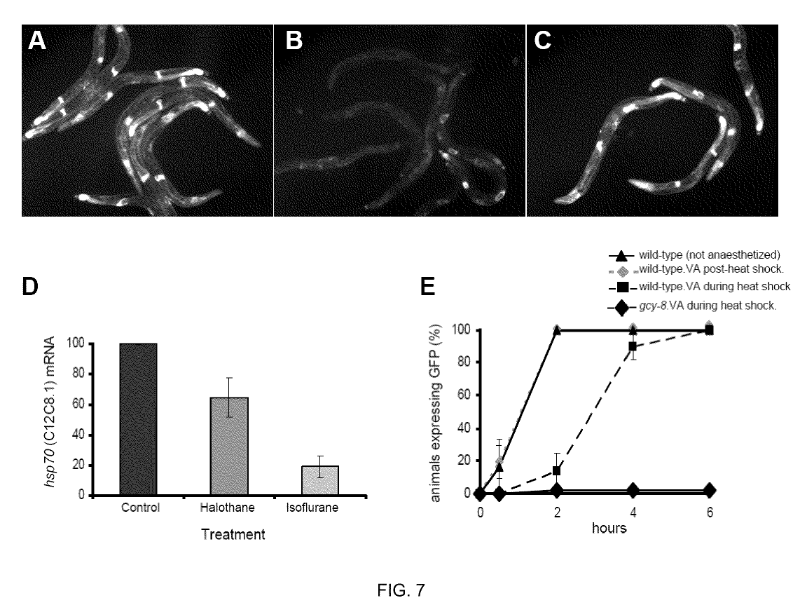

We tested whether the deficiency in HSF-1-dependent heat shock induction of HSPs in the gcy-8 mutants resulted from high constitutive expression of chaperones that negatively autoregulate HSF-1 activity, or other inhibitors HSF-1 (2, 14). This appeared not to be the case: both wild-type and mutant animals expressed similar amounts of constitutive hsp70 (hsp-1) (FIG. 6B), the stress-inducible hsp70s (C12C8.1 and F44E5.4; (8), Table, SI), hsp90 (daf-21) and daf-16 (FIG. 6B). To test whether the gcy-8 mutant animals were deficient in their ability to mount any stress-inducible transcriptional response we exposed animals to another stress, the transition metal cadmium (15), and assayed their ability to activate transcription of two cadmium-responsive genes: hsp70 (C12C8.1) and cdr-1 (15). In contrast to what was observed upon heat shock, both hsp70 and cdr-1 mRNA were similarly increased in wild-type and gcy-8 mutant animals (FIG. 2B) after 3 hours of exposure. Moreover, as for wild-type animals, the cadmium-dependent induction of hsp70 (C12C8.1) was HSF-1-dependent in gcy-8 mutants (FIG. 2C). Thus a deficiency in the AFD neuron does not compromise the molecular machinery required for the stress dependent HSF-1 transcriptional response. The gcy-8 mutant animals are not pre-adapted to stress, but instead are selectively impaired in their ability to induce HSF-1-dependent heat shock gene expression. Given the role of the AFD in sensing ambient temperature, this suggests that AFD signaling is required for heat shock-dependent gene expression. This was further confirmed by transiently and reversibly inhibiting neuronal activity in wild-type animals using the volatile anesthetics (VAs) halothane and isoflurane and observing a concomitant inhibition of hsp70 (C12C8.1) expression in somatic cells ((8), FIG. 7).

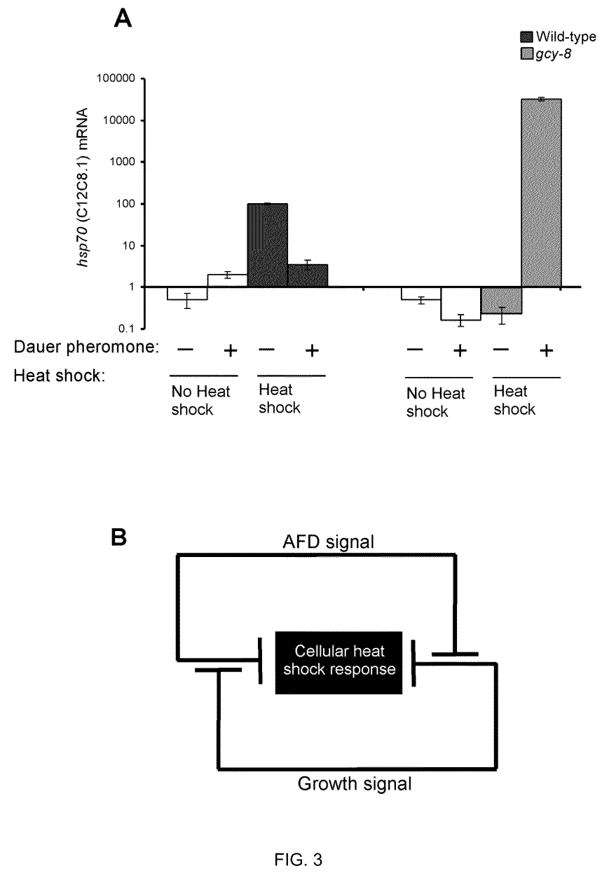

The AFD neurons and associated thermosensory circuitry in C. elegans also regulate thermotaxis behavior, integrating temperature information with environmental signals that modulate growth and metabolism (6). One such potent environmental signal is dauer pheromone: low concentrations of dauer pheromone when animals are at low population densities in the presence of food promote continuous growth; high concentrations signal starvation and alter metabolism (16). AFD mutants show altered sensitivity to dauer pheromone (16, 17). Thus dauer pheromone might also modulate the heat shock response in an AFD-dependent manner integrating the organismal stress response with metabolism. C. elegans raised at low population density initiate transcription of hsp mRNA following heat shock (FIGS. 1 and 3A). However, exposure of wild-type animals to high concentrations of dauer pheromone prior to, or during heat shock decreased the amounts of hsp70 (C12C8.1) mRNA (FIG. 3A). Thus, the heat shock response in C. elegans is affected by the metabolic state and is dampened under conditions that do not support continuous growth and reproduction. In contrast, exposure of gcy-8 mutant animals to dauer pheromone had the opposite effect and induced even higher amounts of hsp70 (C12C8.1) mRNA after heat shock than is normally seen upon heat shock of wild-type animals raised under optimal growth conditions (FIG. 3A).

The opposing effects of dauer pheromone on wild-type and gcy-8 mutant animals can be explained by a model where the heat shock response is regulated at the organismal level by two inputs: the AFD-dependent temperature input, and a metabolic signal that responds to growth conditions (FIG. 3B). Each of these inputs negatively regulates the other and inhibits HSP expression. In wild-type animals, under conditions that support growth, the growth-dependent inhibitory signal is active, and an increase in temperature activates the AFDs, which suppresses the inhibitory signal, thus allowing induction of the heat shock response. In the presence of positive growth signals, but absence of AFD signaling during heat shock (as in gcy-8 mutants), the growth input is not inhibited and the heat shock response is suppressed. The addition of dauer pheromone to wild-type animals suppresses the signal from the growth input, resulting in the inhibition of the cellular heat shock response by AFD signaling. In the absence of both AFD and growth signals, as occurs in the gcy-8 mutant exposed to dauer pheromone, the heat shock response is not inhibited.

The model we propose for the regulation of the heat shock response in C. elegans suggests that cells induce this response in the presence of AFD and growth signals, as in wild-type animals, but also in the complete absence of these regulatory signals, as observed for isolated cells in culture. Thus, neuronal control may allow C. elegans to coordinate the stress response of individual cells, with the varying metabolic requirements of its different tissues and developmental stages. Indeed, neuronal signaling has been shown to modulate cellular homeostasis in C. elegans (18). Because the AFD neurons do not directly innervate any of the downstream tissues in which heat shock gene induction is affected, it is likely that this regulation is mediated through neuroendocrine signaling. The override of the cell-autonomous heat shock response by neuronal circuitry seen for C. elegans may be a common mechanism of regulation in other metazoans. Indeed, HSF-1 in rats can be activated by neuroendocrine signaling from the hypothalamic-pituitary-adrenal axis, in the absence of external stress (19). Thus, the hierarchical organization of regulatory networks may allow organized tissues comprised of heterogenous cell types to establish a highly orchestrated stress response in the metazoan organism.

### FIGURE LEGENDS

FIG. 1. Role of AFD and AIY neurons in the organismal heat shock response. (A) Schematic depiction of genes affecting AFD and AIY function. AFD detects temperature using the cGMP-dependent TAX-4/TAX-2 cyclic-nucleotide gated (CNG) channel. Guanylyl cyclases, gcy-8, gcy-18 and gcy-23 function upstream of tax-4, ODX transcription factor, TTX-1, regulates gcy-8 expression, and AIY function is specified by the LIM homeobox gene, ttx-3. (B) Total hsp70 (C12C8.1) mRNA levels in gcy-8 and ttx-3 mutants relative to wild-type animals, prior to heat shock (pre-H.S.) and 2 hours post-heat shock (post-H.S) at 30° C. and (C) 34° C. for 15 minutes. Time course of total (D) hsp70 (C12C8.1), (E) hsp70 (F44E5.4), and (F) hsp16.2 mRNA accumulation following heat shock (34° C.; 15 minutes) in gcy-8 and ttx-3 mutants relative to wild-type animals. mRNA levels were measured by quantitative reverse-transcriptase-polymerase-chain-reaction (RT-PCR), and normalized to maximal wild-type values. (G) Survival of wild-type, gcy-8, ttx-3 and hsf-1 mutant animals. hsp70 (C12C8.1) promoter-GFP reporter expression in (H) wild-type (I) gcy-8 and (J) ttx-3 mutant animals 2 hours after heat shock (34° C.; 15 minutes). (i) pharynx, (ii) spermatheca, and (iii) intestinal cell. Bar=100 μm.

FIG. 2. Impairment of HSF-1 dependent gene expression in gcy-8 mutants after temperature stress. (A) Total hsp70 (C12C8.1) mRNA, 2 hours post-heat shock (34° C.; 15 minutes), in wild-type and gcy-8 mutant animals subjected to RNAi-mediated knockdown of hsf-1 or daf-16. mRNA levels were measured by quantitative RT-PCR and normalized to wild-type values on control RNAi. (B) Total hsp70 (C12C8.1) and cdr-1 mRNA levels in wild-type and gcy-8 mutants following cadmium stress (8). mRNA levels were measured by quantitative RT-PCR, and normalized to wild-type values. (C) Total hsp70 (C12C8.1) mRNA levels in cadmium-treated wild-type animals and gcy-8 mutants subjected to RNAi-mediated knockdown of hsf-1. mRNA levels were measured by quantitative RT-PCR and normalized to wild-type and gcy-8 values on control RNAi.

FIG. 3. AFD-dependent regulation of the cellular heat shock response is modulated by metabolic signals. (A) Total hsp70 (C12C8.1) mRNA levels prior to, and 2 hours after heat shock (34° C.; 15 minutes) in wild-type and gcy-8 mutant animals grown at low population densities, or exposed to dauer pheromone 10 minutes prior to, and during the 2 hours of recovery following heat shock. Note semi-logarithmic scale. mRNA levels were measured by quantitative RT-PCR, and normalized to maximal wild-type values. (B) Model depicting the regulation of the cellular heat shock response by AFD-dependent signaling of temperature and dauer pheromone-dependent signaling of growth conditions.

### Materials and Methods

C. elegans Strains

The following C. elegans strains were used: C. elegans Bristol wild-type N², gcy-8 (oy44) IV, gcy-23 (nj37) IV (1), PR678 tax-4 (p678) III (2), PR767 ttx-1 (p767) V (3), FK134 ttx-3 (ks5) X (4), PS3551 hsf-1 (sy441) I (5), ocr-2 (ak47) IV (6), unc-54 (e1092) I (7), and the hsp70 (C12C8.1) promoter GFP heat shock reporter (8). The hsp70 (C12C8.1) promoter GFP heat shock reporter transgenic strain also contained a rol-6 co-injection marker: animals displayed a roller phenotype, and adults are impaired in motility. To generate gcy-8 and ttx-3 mutant animals carrying the hsp70 (C12C8.1) promoter GFP heat shock reporter, gcy-8 (oy44) IV or ttx-3 (ks5) X animals were crossed with those carrying the GFP reporter and progeny were selected for their roller phenotype. The genotypes were verified by PCR when necessary. gcy-8 (oy44) IV primers that have been previously described in (1) were used.

gcy-8 (oy44) and gcy-23 (nj37) were obtained from Dr. I. Mori, Nagoya University, Nagoya, Japan. The remaining strains were obtained from the Caenorhabditis Genetics Center (CGC).

All the thermosensory mutations (gcy-8 (oy44), ttx-3 (ks5), gcy-23 (nj37), and tax-4 (p678)) except ttx-1 (p676) are either loss of function mutations or protein nulls, as specifically described below.

The gcy-8 (oy44) mutation is a deletion affecting the kinase homology and cyclase regions of the guanylyl cyclase protein, and is likely to be a functional null (1). The AFD-specific expression of gcy-8 gene product was established by expressing transcriptional fusion constructs (gcy-8promoter::GFP fusion) in C. elegans (9). The gcy-8 promoter chosen for these studies extended approximately 2 kb upstream until the nearest predicted gene. Subsequently, AFD-specific expression of gcy-8 has been confirmed by studies that have used microarrays and expression profiling to identify neuronally expressed genes (10, 11). The gcy-8 protein fusion, made using full length genomic DNA fused to GFP, has been expressed in C. elegans and localizes exclusively to the sensory endings of AFD neurons (1). Thermotaxis assays suggest that while the gcy-8 (oy44) mutation alone has a very mild cryophilic phenotype; along with mutations in the other guanylyl cyclases expressed in the AFD, such as gcy-23, it shows a thermotaxis defect (1). The gcy-8 protein fusion when expressed in this background rescues this thermotaxis defects (1).

The ttx-3 (ks5) mutation is a point mutation in a splice donor site within the gene, and does not appear to express protein (12). The animals are cryophilic, mimicking ablations in the AFD or AIY neuron.

The gcy-23 (nj37) mutation is a deletion within the coding sequence and also thought to be a functional null (1).

The tax-4 (p678) mutation (2) causes the conversion of glutamine (82) to a stop codon in the region near the NH₂-terminus, and is therefore expected to be a null mutation.

The ttx-1 (p767) alters splicing in some but not all transcribed messenger RNAs, and is likely not a molecular null. (3). However, the mutants show cryophilic thermotaxis behavior suggesting that AFD function is affected in a manner similar to that in the other mutants.

The ocr-2 (ak47) mutation does not affect the thermosensory function of the AFD neuron, but instead affects the sensory function of the four other neurons: ADF, AWA, ASH and ADL (6).

### Growth Conditions

The general methods for growing C. elegans were as described (13). The quality of bacterial food, and population densities of C. elegans, greatly influenced the outcome of all experiments so extreme care was taken to consistently expose the different C. elegans strains to bacterial lawns similarly grown and to maintain the animals at low population densities throughout their development and prior to and during the experiments.

The bacteria Op50 was used for feeding C. elegans (13). Standard NGM plates (13) of 6 cm in diameter with the thickness of the agar set at 6 mm ensured similar rates of heat transfer. Plates were seeded with 200-500 μl of a stationary phase culture of Op50 grown in LB broth. The bacteria were allowed to establish a dense bacterial lawn at room temperature for 48 hours and no more than 72 hours before being plated with the appropriate C. elegans strains. Care was taken to prevent contamination with other bacteria.

To ensure that C. elegans used in experiments were exposed to low population densities and optimal growth conditions, five animals in the L4 stage were placed on Op50 seeded plates, allowed to reproduce at 20° C., and their progeny were allowed to develop for 48 to 72 hours before being transferred onto new, similarly seeded NGM plates for use in experiments. Typically 10 L4 progeny, grown as described, were transferred per plate, allowed to develop into adults for 22-24 hours at 20° C. and corresponded to one sample in an experiment. Sufficient ‘N’ values were obtained by increasing the number of plates, and not by increasing the number of animals per plate.

### Heat Shock Protocol

C. elegans strains were grown as described above, and all animals were heat shocked at a population density of 10 adults per plate. Sufficient N values were obtained by repeating each heat shock experiment a minimum of 3 times, with 3 samples of 10 animals per plate, per experiment. Heat shock at 30° C. or 34° C., for 15 minutes on agarose plates was achieved by sealing plates with parafilm, further sealing within zip-lock bags, and immersing in a water bath equilibrated to the appropriate temperature. Following heat shock, the parafilm was removed and the animals were allowed to recover at 20° C. for the course of the experiment.

We determined that this heat shock procedure resulted in the exposure of the somatic cells of both the wild-type and mutant animals to the same temperature, and that thermotaxis differences between the wild-type and thermosensory mutants did not confound our interpretation of data, by: (a) ensuring that the temperature equilibrated rapidly across the agarose plates and that there were no temperature gradients, (b) ensuring that the surface area of the mutant and wild-type animals was comparable and, (c) assaying heat shock gene induction of the wild-type and thermosensory mutants gcy-8 and ttx-3 in the rot-6 genetic background that abolished their ability to migrate across the plate.

In order to determine the rate of equilibration of the agarose plates, we directly measured the rate of temperature increase at 10 random but well spaced points within the plate using a thermocouple (Fluke, 51 II Thermometer, Byram Labs, Everett, Wash.). Consistent and rapid equilibration of the heat shock temperature was attained at all points across the plate within the duration of heat shock (FIGS. 4A and 4B). The heat shock temperature of 30° C. was attained by 6 minutes (FIG. 4A), and 34° C. was attained at all points by 7 minutes (FIG. 4B). The temperature did not fluctuate within the range of detection of the thermocouple (0.01° C.) during the remainder of the heat shock (FIGS. 4A and B).

We examined whether there were thermal gradients formed across the plate using a 0.008″ thick thermochromic Liquid Crystal (LC) ink plastic film (Edmund Scientific, Liquid Crytal Mylar sheets, Calatog #3072372; FIG. 4C-F). The LC sheet was calibrated using the thermocouple and produced color at wavelength in the red range at 30° C. (FIG. 4D), and blue at 34° C. (FIG. 4E). The LC sheets were then cut to the size of the agarose plates (6 cm diameter) and applied onto the surface of the plates that had been seeded with Op50 bacteria. These plates were then subjected to the heat shock protocol described above and photographed immediately after. Plates that were immersed in the 30° C. water bath turned red (FIG. 4D), while those immersed in a 34° C. water bath, as described above, turned blue (FIG. 4E). This latter temperature was used for the majority of the experiments. We ensured that the LC sheets were indeed capable of detecting temperature gradients by applying a gradient of 25-34 degrees to one of the plates, and obtaining a gradient of color change (FIG. 4E).

To confirm that the mutant and wild-type animals subjected to the temperature stress had a comparable surface area, we measured the surface area of 30 images of wild-type, gcy-8 (oy44) and ttx-3 (ks-5) animals using Image J. Both the surface area measurements (depicted as pixel number) and the variation seen amongst different animals of each strain were very similar between wild-type and thermosensory mutant animals (FIG. 4G).

To ensure that that motility across the agarose did not affect heat shock dependent gene induction, we compared endogenous hsp70 (C12C8.1) mRNA levels in the hsp70p (C12C8.1):: GFP; rot-6 heat shock reporter strains that were wild-type with those carrying a mutation in their gcy-8 and ttx-3 genes.

### RNA Extraction and Quantitative RT-PCR

mRNA was prepared using the “ABSOLUTELY RNA® Nanoprep Kit” (Stratagene, Catalog #400753). The manufacturer's protocol was adapted to achieve maximal lysis of worms. Briefly, 5-10 adult animals were picked either from the control or experimental plates into 100 μl of buffer made up by mixing 7 μl 13-ME (instead of the recommended 0.7 μl 13-ME), with 100 μl Lysis Buffer provided by the manufacturer. The suspension was subjected to numerous cycles of freeze-thawing in liquid nitrogen and vortexing until the animals were completely lysed. RNA was then purified as detailed in the manufacturer's protocol. mRNA was reverse transcribed using the ISCRIPT™ cDNA Synthesis Kit (Bio-Rad, Catalog #170-8891). Quantitative PCR was performed using iQ™ SYBR® Green Supermix (Bio-Rad, Catalog #170-8880), in the iCycler system (Bio-Rad) at a 25 μl sample volume, in thin wall 200 μl PCR plates (Cat. No. 223-9441) sealed with the optical quality sealing tape (Cat. No. 223-9444).

The relative amounts of hsp mRNA were determined using the Comparative C_{T} Method for quantitation (14). The levels of hsp mRNA levels within an experiment were determined relative to actin mRNA, which was used as the internal control. The range of input of RNA was determined using serial dilutions of the cDNA that yielded a C_{T} value of <30, for both the target cDNA and actin was used in all experiments. This typically corresponded to 1 μl of the total cDNA obtained per sample. C_{T} values were obtained in triplicate for each sample (technical triplicate), and three samples were used per experiment. Each experiment was then repeated a minimum of three times. All relative changes of hsp mRNA in the mutant strains were normalized to maximal wild-type values, except where otherwise noted.

The heat shock time course data shown in FIG. 4D-F are representative for the indicated hsp genes over 6 hours. Each time point depicts the average of three technical triplicates from three samples, in one experiment. During the course of recovery, different experimental replicates, which reflected different biological samples, all showed a consistent decrease in the maximal induction of hsp mRNA in the mutant strains compared to wild-type values. However, the levels of hsp genes between different biological samples were variable, especially during the later time points.

### Thermotolerance Assay

Thermotolerance assays were conducted on wild type N2, gcy-8 (oy44) IV, ttx-3(ks5) X, and the hsf-1 (sy441) I animals grown as described above. Ten samples, each containing ten adult animals per plate, were used for one thermotolerance experiment, and three repetitions of the experiment were performed to obtain substantial ‘N’ values. Thermotolerance assays were conducted by immersing animals in a 35° C. water bath for 7-9 hours. This duration of exposure was required to obtain 50% death of the wild-type N2 animals, and survivors were scored approximately 12 hours after recovery at 20° C.

### RNAi Experiments

Escherichia coli strain HT115 (DE3) harboring the appropriate dsRNA expressing plasmid from the genomic RNAi library (J. Arhinger) were grown overnight in LB broth containing ampicillin (100 μg/ml) and tetracycline (12.5 μg/ml). 200-500 μl of bacteria was seeded onto NGM plates containing ampicillin (100 μg/ml) and tetracycline (12.5 μg/ml) and 0.4 mM Isopropyl β-d-thiogalactosidase. Care was taken to ensure that the plates grew a healthy lawn of RNAi bacteria by allowing the bacteria to grow for 2-4 days prior to use. For each RNAi experiment, 10 animals were singled onto the RNAi plates as L4 larvae, and allowed to develop for 22-26 hours into adults prior to use. In all cases, the knock-down of the appropriate RNA was confirmed by RT-PCR (not shown). The RNAi constructs used were directed against either hsf-1 or daf-16. Escherichia coli strain HT115 (DE3) harboring the RNAi plasmid vector L440 alone was used as the control.

### Cadmium Stress Experiments

Sterile-filtered cadmium chloride was added to a final concentration of 50 μM to standard, autoclaved, NGM medium, and used to make plates. OP50 was seeded onto the plates as described above. To assay cadmium-responsive gene expression, wild-type N2 and gcy-8 mutant animals were grown on regular NGM plates in the absence of cadmium as described above, and following their development to adults, 10 animals were transferred onto the cadmium-plates for a duration of 3 or 16 hours. These animals were then harvested for quantitative RT-PCR. The levels of hsp70 (C12C8.1) mRNA induction in both the wild-type and gcy-8 mutant animals after 3 hours is indicated in the text (FIG. 2B). hsp70 (C12C8.1) mRNA was further induced more than 10-fold after 16 hours of exposure.

To conduct RNAi experiments aimed at assessing the effects of hsf-1 knock-down on the induction of cadmium-responsive genes, animals were exposed to both dshsf-1 and cadmium as L4 larvae for 24-28 hours. This was done by growing the animals on RNAi plates containing cadmium, and seeded with RNAi bacteria harboring the dshsf-1 plasmid. Animals were harvested for RT-PCR 28 hours after being placed on the RNAi plates, and knock-down of hsf-1 RNA was confirmed by RT-PCR.

### Anesthesia Experiments

The VA anesthetics used were 2-Bromo-2-chloro-1,1,1-trifluoroethane (Halothane, Fluka, catalog #16730) and Isoflurane (Webster Veterinary NDC#14043-220-05). VA anesthetics were delivered as follows: lids of 1.5 ml eppendorf tubes were cut off, VA was pipetted into the lids, and the lids containing VAs were placed onto plates containing 10 adult wild-type or gcy-8 animals grown as described, and the plates were immediately sealed with parafilm. To inhibit neuronal signaling during the course of heat shock, the VA containing lids were placed on plates 5 minutes prior to the heat shock, and retained during the heat shock treatment of 34° C. for 15 minutes. The lids were then removed 10 minutes post-heat shock during recovery at 20° C., when the plates had equilibrated to 20° C. To inhibit neuronal signaling following the administration of heat shock, animals were heat shocked, and then the lids containing the same volume of VA was placed onto plates 20 minutes post-heat shock, and removed after 30 minutes.

To control for non-specific effects of paralysis on heat shock gene induction, we measured hsp70 (C12C8.1) mRNA levels in animals with functional AFDs, but paralyzed due to a partial deletion in their myosin gene (unc-54). These animals showed normal heat shock induction of hsp70 (C12C8.1) (mRNA levels in unc-54 (e1092) animals relative to wild-type induction: wild-type=1.00, unc-54 (e1092)=1.6±0 5, measures 2 hours post-34° C. for 15 minutes).

As has been described before, the effect of the VA is extremely variable in any given population of C. elegans (15, 16) and is also influenced by other environmental factors, such as population density, to which the animals are exposed (15). Therefore it was necessary to titrate the amount of VA used for each experiment. The volume of VA used was chosen as that which inhibited the movement of 100% of the animals on a plate within the first 15 minutes following exposure to the VA, did not cause any death over the course of the experiment, and did not result in all the animals consistently moving off the bacterial lawn following recovery from VA. Using C. elegans grown as described above, this corresponded to 5-15 μl for halothane and 10-25 μl for isoflurane. Animals were considered to have recovered from the effects of the anesthesia when they were actively moving on plates, and this occurred within 1 hour following VA exposure, when 100% of the animals had recovered.

### Dauer Pheromone Experiments

The effect of dauer pheromone on the heat shock response was tested using DAUMONE ((17) KDR Biotech. Co. Ltd. Cat # DA-1-010). Daumone stocks were prepared by dissolving daumone in ethanol (320 μg in 100 μl). C. elegans were grown as described above, and 5-10 minutes prior to heat shock, 10-50 μl of daumone was spotted onto the OP50 plate containing 10 adult C. elegans. Care was taken not to let the daumone touch the animals. 50 μl ethanol was used as controls. The animals were allowed to recover from heat shock on the same plates in the presence of daumone, after which they were harvested for mRNA.

FIG. 4 shows that the heat shock procedure resulted in the exposure of the somatic cells of both the wild-type and mutant animals to the same temperature.

FIG. 5 shows that the gcy-8 or ttx-3 mutant animals continued to be impaired in hsp70 (C12C8.1) promoter GFP reporter construct expression 24 hours following heat shock.

FIG. 6 shows that the gcy-8 and ttx-3 mutant animals do not express less hsf-1 compared to wild-type animals. In fact, gcy-8 and ttx-3 animals perhaps express more hsf-1 mRNA relative to wild-type animals. The gcy-8 animals do not express higher constitutive amounts of chaperones that negatively autoregulate HSF-1 activity, or other inhibitors HSF-1 (18, 19). Thus these explanations do not sufficiently explain the diminished heat shock dependent expression of HSP mRNA in the thermosensory mutant animals.

FIG. 7 shows that cellular heat shock response is neuronally regulated. If AFD signaling is required for the heat shock response the inhibition of neuronal activity in wild-type animals should inhibit the transcription of genes encoding HSPs, mimicking the effect of AFD mutations. We used the volatile anesthetics (VAs) halothane and isoflurane which inhibit synaptic transmission to transiently and reversibly inhibit neuronal activity (16). Wild-type animals exposed to VAs for the full duration of the heat shock showed a marked decrease in hsp70 (C12C8.1) expression 2 hours after recovery (FIG. 7). This was evident in the reduced levels of hsp70 (C12C8.1) promoter GFP reporter expression (FIG. 7A-C), and the fraction of animals expressing GFP (20% versus 100% control; FIG. 7E), providing independent corroboration that the cellular heat shock response is neuronally regulated.

Surprisingly, wild-type animals inhibited in the induction of hsp70 expression at 2 hours recovered hsp70 mRNA expression as the anesthetic effect dissipated (FIG. 7E). This recovery required the normal functioning of the AFD neuron: gcy-8 mutants subjected to VAs did not recover GFP reporter expression even after the anesthesia wore off (FIG. 7E). These data confirm that the expression of hsp70 (C12C8.1) mRNA in somatic cells requires active gcy-8-dependent neuronal signaling. In addition they may provide some clues into the mechanism of neuronal control of the cellular heat shock response, in which binding of HSF-1 to its promoter still requires an active neuronal signal to activate transcription.

### FIGURE LEGENDS

Legend to FIG. 4: Both the wild-type and thermosensory mutant animals are exposed to the same temperature during heat shock. (A) The rate of temperature increase, averaged across 10 random, well spaced points on a 6 mm thick agarose plate used for the heat shock experiments when plates were transferred from 20° C. to a 30° C. water bath for 15 minutes, and (B) when plates were transferred to a 34° C. water bath for 15 minutes. (C) A photograph of a 0.008″ thick thermochromic Liquid Crystal (LC) ink plastic film which changes colour to indicate temperature (red=30° C. and blue=34° C.) when applied to the surface of an agarose plate at 20° C. (D) The LC film after applied to the surface of an agarose plate immersed uniformly in a 30° C. water bath for 15 minutes. (E) The LC film after being applied to the surface of an agarose plate immersed uniformly in a 34° C. water bath for 15 minutes. (F) The LC sheet when applied to the surface of an agarose plate exposed to a temperature gradient of 25-34° C. for 15 minutes by immersing half of the plate in the 34° C. water bath, while the other half remained at room temperature of 25° C. (G) The surface area of 30 images each of wild-type, gcy-8 and ttx-3 thermosensory mutant animals.

Legend to FIG. 5: hsp70 (C12C8.1) promoter-GFP reporter expression in (A) wild-type (B) gcy-8 and (C) ttx-3 mutant animals 24 hours post-heat shock (34° C.; 15 minutes).

Legend to FIG. 6: A) Basal hsf-1 mRNA levels in wild-type and gcy-8 and ttx-3 mutants. (B) Basal mRNA levels of daf-16, hsp90 (daf-21) and hsp70 (hsp-1), in wild-type and gcy-8 mutants. mRNA levels were measured relative to the wild-type strain, by quantitative RT-PCR.

Legend to FIG. 7

Requirement of active neuronal signaling for heat shock gene expression. hsp70 (C12C8.1) promoter-GFP reporter expression assayed 2 hours post-heat shock in (A) control, non-anesthetized wild-type worms, (B) wild-type worms anesthetized with VA during heat shock, and (C) wild-type worms anesthetized with VA following heat shock. (D) Total hsp70 (C12C8.1) mRNA levels 2 hours post-heat shock in control non-anesthetized worms, and worms anesthetized with VAs (halothane and isoflurane) (pair-wise t-test; P value=0.001 and 0.0001 respectively). (E) Percentage of wild-type or gcy-8 mutant animals expressing the hsp70 (C12C8.1) promoter-GFP reporter at various times post-heat shock. Heat shock in all experiments was 34° C. for 15 minutes. mRNA levels were measured by quantitative RT-PCR and normalized to wild-type values.

TABLE SI

| | | 30° C. | 34° C. |
|---|---|---|---|
| Strain | Baseline | heat shock | heat shock |
| Wild-type | 1.4 ± 0.3 | 100.0 | 100.0 |
| gcy-23 | 1.1 ± 0.3 | 18.4 ± 0.6 | 10.2 ± 0.7 |
| gcy-8 | 3.6 ± 2.3 | 21.9 ± 12.0 | 12.2 ± 8.4 |
| tax-4 | 0.9 ± 0.3 | 40.1 ± 9.7 | 43.1 ± 16.8 |
| ttx-1 | 0.9 ± 0.0 | 42.0 ± 8.8 | 36.0 ± 12.8 |
| ttx-3 | 2.8 ± 2.0 | 17.9 ± 8.4 | 6.7 ± 5.0 |

Legend to Table SI

hsp70 (C12C8.1) mRNA levels in wild-type, gcy-8 (oy44) IV, gcy-23 (nj37) IV (1), PR678 tax-4 (p678) III (2), PR767 ttx-1 (p767) V (3) and FK134 ttx-3(ks5) X (4), prior to heat shock (column 2), 2 hours post-heat shock at 30° C. for 15 minutes (column 3), and 2 hours post-heat shock at 34° C. for 15 minutes (column 4). mRNA levels were measured by quantitative RT-PCR. Baseline hsp70 (C12C8.1) mRNA values were normalized to the maximal wild-type induction following the 34° C. heat shock. hsp70 (C12C8.1) mRNA values following heat shock at either temperature was normalized to wild-type values at that temperature. In addition, hsp70 (C12C8.1) mRNA levels in ocr-2 (ak47) mutant animals=90±20.2, 2 hours post-heat shock at 34° C. for 15 minutes. hsp70 (C12C8.1) mRNA levels in the heat shock reporter transgene containing animals, 2 hours post-heat shock at 34° C. for 15 minutes.: wild-type hsp70p (C12C8.1)::GFP; (rol-6)=100, gcy-8; hsp70p (C12C8.1)::GFP; (rol-6)=4.7±2.1 and ttx-3; hsp70p (C12C8.1)::GFP; (rol-6)=26.5±13.2. mRNA levels were measured relative to the wild-type strain. mRNA levels in all cases was measured by quantitative RT-PCR.

### REFERENCES

1. S. Lindquist, E. A. Craig, Annu Rev Genet. 22, 631 (1988).
2. R. I. Morimoto, Genes Dev 12, 3788 (1998).
3. E. G. Stringham, E. P. Candido, J Exp Zool 266, 227 (1993).
4. J. H. Feder, J. M. Rossi, J. Solomon, N. Solomon, S. Lindquist, Genes Dev 6, 1402 (1992).
5. D. A. Clark, C. V. Gabel, H. Gabel, A. D. Samuel, J Neurosci 27, 6083 (2007).
6. I. Mori, Annu Rev Genet. 33, 399 (1999).
7. H. Inada et al., Genetics 172, 2239 (2006).
8. Supporting Online Material is available on Science Online.
9. T. P. Snutch, M. F. Heschl, D. L. Baillie, Gene 64, 241 (1988).
10. I. Sokolchik, T. Tanabe, P. F. Baldi, J. Y. Sze, J Neurosci 25, 1015 (2005).
11. C. D. Link, J. R. Cypser, C. J. Johnson, T. E. Johnson, Cell Stress Chaperones 4, 235 (1999).
12. Y. M. Hajdu-Cronin, W. J. Chen, P. W. Sternberg, Genetics 168, 1937 (2004).
13. J. F. Morley, R. I. Morimoto, Mol Biol Cell 15, 657 (2004).
14. A. L. Hsu, C. T. Murphy, C. Kenyon, Science 300, 1142 (2003).
15. Y. Cui, S. J. McBride, W. A. Boyd, S. Alper, J. H. Freedman, Genome Biol 8, R122 (2007).
16. P. J. Hu, WormBook, ed. The C. elegans Research Community, WormBook, doi/10.1895/wormbook.1.144.1, http://www.wormbook.org., 1 (2007).
17. J. W. Golden, D. L. Riddle, Proc Natl Acad Sci USA 81, 819 (1984).
18. S. M. Garcia, M. O. Casanueva, M. C. Silva, M. D. Amaral, R. I. Morimoto, Genes Dev 21, 3006 (2007).
19. T. W. Fawcett, S. L. Sylvester, K. D. Sarge, R. I. Morimoto, N. J. Holbrook, J Biol Chem 269, 32272 (1994).
20. We thank the Morimoto lab members for discussions and comments. We acknowledge T. Stiernagle and the Caenorhabditis elegans Genetics Center, supported by a grant from NIGMS and the International Consortium, for providing strains. We thank Dr. I. Mori at Nagoya University, Nagoya, Japan for the gcy-8(oy44) strains. These studies were supported by grants to R.I.M from the N.I.H (NIGMS and NIA), the Huntington's Disease Society of America Coalition for the Cure, and the Daniel F. and Ada L. Rice Foundation.
S1. H. Inada et al., Genetics 172, 2239 (April, 2006).
S2. H. Komatsu, I. Mori, J. S. Rhee, N. Akaike, Y. Ohshima, Neuron 17, 707 (October, 1996).
S3. J. S. Satterlee et al., Neuron 31, 943 (Sep. 27, 2001).
S4. O. Hobert et al., Neuron 19, 345 (August, 1997).
S5. Y. M. Hajdu-Cronin, W. J. Chen, P. W. Sternberg, Genetics 168, 1937 (December, 2004).
S6. I. Sokolchik, T. Tanabe, P. F. Baldi, J. Y. Sze, J Neurosci 25, 1015 (Jan. 26, 2005).
S7. N. J. Dibb et al., J Mol Bio1183, 543 (Jun. 25, 1985).
S8. J. F. Morley, R. I. Morimoto, Mol Biol Cell 15, 657 (February, 2004).
S9. S. Yu, L. Avery, E. Baude, D. L. Garbers, Proc Natl Acad Sci USA 94, 3384 (Apr. 1, 1997).
S10. M. E. Colosimo et al., Curr Biol 14, 2245 (Dec. 29, 2004).
S11. S. E. Von Stetina et al., Genome Biol 8, R135 (2007).
S12. Z. Altun-Gultekin et al., Development 128, 1951 (June, 2001).
S13. S. Brenner, Genetics 77, 71 (May, 1974).
S14. A. biosystems, http://www.appliedbiosystems.com/support/apptech/ (2004).
S15. B. van Swinderen, L. B. Metz, L. D. Shebester, C. M. Crowder, Genetics 161, 109 (May, 2002).
S16. P. G. Morgan, M. Sedensky, P. M. Meneely, Proc Natl Acad Sci USA 87, 2965 (April, 1990).
S17. P. Y. Jeong et al., Nature 433, 541 (Feb. 3, 2005).
S18. R. I. Morimoto, Genes Dev 12, 3788 (Dec. 15, 1998).
S19. A. L. Hsu, C. T. Murphy, C. Kenyon, Science 300, 1142 (May 16, 2003).

### Example 2

### Neuronal Regulation of Chaperone Expression

Example 1 and Prahlad et al. (2008). Regulation of the Cellular Heat Shock Response in Caenorhabditis elegans by Thermosensory Neurons. Science 320(5877): 811-814 showed that the heat shock response in C. elegans is regulated in a cell non-autonomous manner by the neurosensory circuitry that detects temperature. Two thermosensory (AFD) neurons in C. elegans regulate the HSF1-dependent transcription of heat shock genes throughout the organism. These results showed that signaling by the thermosensory neurons modulate HSF1-dependent transcriptional activity in response to heat shock and nutritional signals. In AFD-deficient animals, the expression of heat shock genes could be induced by exposure to cadmium, which suggests specificity in stress signaling. Consequently, we proposed that neuronal signaling is an important component in the regulation of chaperones and other cytoprotective mechanisms and affords a novel mechanism for the integration of the stress response with organismal growth and metabolism.

The following additional aspects of the study have been investigated:

1) What are the AFD-Dependent Signaling Pathways that Cell Non-Autonomously Affect HSF-1 Activation?

The AFD thermosensory neurons do not directly innervate the non-neuronal cells where heat shock-dependent hsp70 mRNA is induced. We therefore hypothesized a role for neuroendocrine signaling. To identify the signaling pathways involved, we initiated a candidate RNAi screen and reduced the expression of genes corresponding to the three major neuroendocrine pathways of C. elegans and examined the consequences on the heat shock induction of hsp70 mRNA throughout the organism. Animals expressing the heat-shock inducible hsp70p::GFP transgene were used for the screen. In order to address the refractory nature of C. elegans neurons to RNAi screen, these animals were crossed into the mutant rrf-3 background which renders the animals hypersensitive to RNAi. The candidates selected for this screen belong to the insulin like (IL)/insulin like growth factor (IGF) signaling pathway, the transforming factor beta (TGF-β), and the steroid hormone pathway. These ligands are expressed by the AFD neuron and/or neurons that form synaptic connections onto the AFD.

Animals were grown up to L4 stage under normal growth conditions, transferred onto RNAi bacteria at the L4 stage, and 24 hours later their heat shock response (34° C. for 15 minutes) was assessed by screening for inducible GFP expression at 24 hours following heat shock. Due to the imperfect penetrance of RNAi even in the sensitized background and the resulting variability, we scored as hits, only those genes whose RNAi markedly reduced GFP expression consistently in at least three separate experiments and observed in 50% or more of the animals. These hits were then slated to be confirmed using mutants in any gene of interest. The following candidate genes were tested:

TGF-beta signaling pathway (11 genes): ligands: daf-7, dbl-1, tig-2, unc-129, dbl-1; -receptors: daf-1, daf-4, sma-6; transcription factors: daf-5, sma-9, lin-31.

Insulin-like signaling pathway (6 genes): ins-2, ins-7, ins-11, ins18, ins-21 and ins-23.

Steroids hormones pathway (5 genes): daf-9 and daf-12; genes involved in cholesterol transport ncr-1 and ncr-2; and genes involved in steroid hormone metabolism let-767.

Neuropeptides (24 genes): nlp-2, nlp-3, nlp-4, nlp-5, nlp-6, nlp-7, nlp-8, nip-11, nlp-12, nip-14, nlp-16,nlp-17, nip-18, nlp-20, nip-21, nlp-22, nlp-23, nlp-24, nlp-25, nip-26,nlp-28, nlp-30, nip-31, nlp-32.

Nuclear hormones receptors (6 genes): nhr-8, nhr-23, nhr-25, nhr-41, nhr-67, nhr-38.

Among the 52 candidates tested, we have identified the following candidate genes which, when knocked down reduce heat shock induction of hsp70p:GFP throughout the animal:

1) Candidates from the IL/IGF-signaling pathway: ins-2, ins-18, and ins-23;
2) Candidates from the TGF-β-signaling pathway: dbl-1, daf-4, sma-9, tig-2, unc-129, lin-31;
3) Candidates from the steroid hormone signaling pathway: daf-9, daf-12; and
4) Neuropeptide like molecules: nlp-4, nlp-5, nlp-7, nlp-21, nlp-22.

This candidate RNAi screen identified ligands belonging to all the three major neuroendocrine signaling pathways as modulators of heat shock-dependent hsp70 mRNA expression. The genes identified are expressed not only in the AFD neuron (dbl-1, nhr-38, nlp-18, nlp-23, ins-2, ins-18 and ins-23), but also in the amphid neurons that communicate with the AFD neuron by gap junction or synapses (ins-7, nlp-6, nlp-3, nlp-4). In addition, some of the gene products identified in this screen are expressed in other cell types such as the neuroendocrine cell (daf-12) and intestinal and hypodermal cells (e.g. daf-4, sma-9), suggesting that the cell non-autonomous regulation of HSP expression upon heat shock may not be under the sole regulation of any single pathway, but once initiated by the AFD, may be transmitted or amplified throughout the organism involving numerous tissues and pathways.

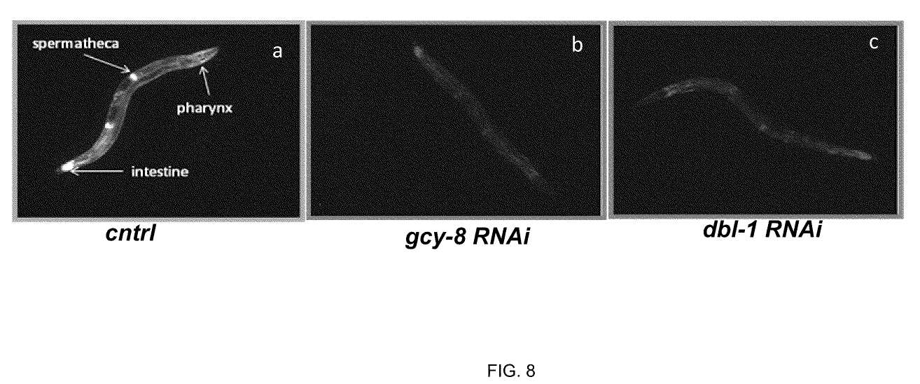

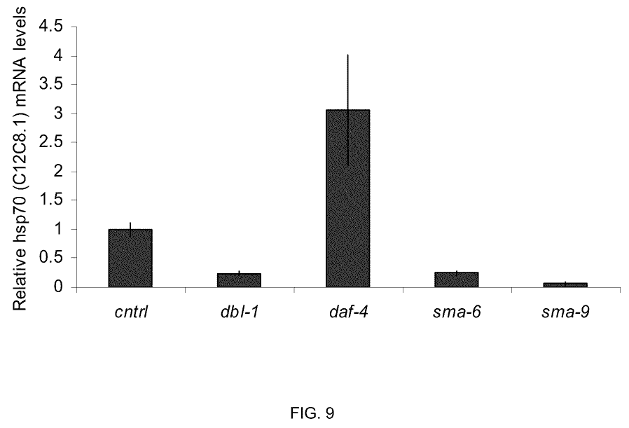

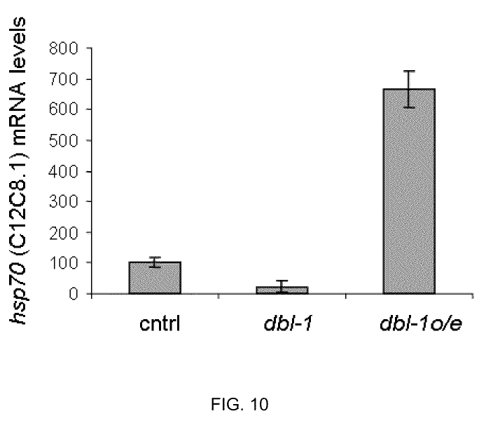

The effects of the knock down of the TGF-β ligand dbl-1 on the heat shock response was pursued in greater detail (FIG. 8). TGF-β signaling pathway consists of a large family of secreted peptide growth factors in metazoans that play a role in growth and development. The canonical TGF-β signal transduction pathway is comprised of two transmembrane ser/thr kinase receptors and 2 or 3 intracellular smad signals. In C. elegans, four TGF-beta ligands have been identified by homology: daf-7, dbl-1, tig-2, and unc-129 (Savage-Dunn C., 2005). DBL-1 is the TGF-beta related ligand for the Sma/mab pathway and is the C. elegans homologue of the bone morphogenic protein (BMP-4). The dbl-1 gene regulates body size and male tail morphogenesis. DBL-1 signal is transduced by SMA-6 type I receptor, DAF-4 type II receptor, SMA-2, SMA-3 and SMA-4 Smads and SMA-9 schnurri. The reduction of hsp70 (C12C8.1) induction upon knockdown of dbl-1 mRNA by RNAi (FIGS. 9 and 10) was further confirmed by RT-PCR. In addition, animals harboring extra copies of the dbl-1 gene induced higher than normal levels of heat shock-dependent hsp70 (C12C8.1) (FIG. 10). Finally, animals carrying mutations in, and/or subjected to RNAi against genes of, the DBL-1 signaling pathway also showed aberrant hsp70 (C12C8.1) expression following heat shock (FIG. 9). These preliminary data suggest that the DBL-1 pathway is involved in regulation of hsp70 (C12C8.1) expression at the level of the organism. We are currently in the process of confirming these results and dissecting the involvement of the TGF-beta ligand, dbl-1 and its associated genes in the cell non-autonomous regulation of the heat shock response.

### (2) Does Neuronal Signaling Confer the Specificity of Stress Responses?

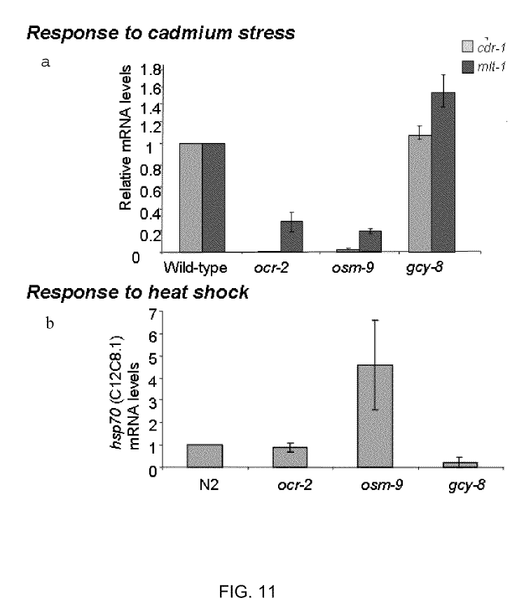

We have examined whether signaling by sensory neurons imparts information regarding the specificity of the stress signal sensed by the organism. To address this, we have investigated whether sensory neurons known to be required for directing an aversive response of C. elegans to the heavy metal cadmium regulate cadmium-dependent gene induction in the organism. Animals harboring mutations in osm-9, a TRPV channel, and ocr-2 have impaired function of their AWA, ASH, ASE, and ADL neurons required for sensing the presence of cadmium ions in the environment. osm-9 and ocr-2 animals are deficient in their ability to upregulate the expression of the cadmium responsive genes, cdr-1 and mtl-1, that are expressed in the intestine upon exposure to cadmium. The mutations in these metal sensory pathways do not interfere with the ability to respond to heat shock. Conversely the gcy-8 (thermosensory deficient) adult animals that are impaired in the heat shock response can induce cdr-1 and mtl-1 upon exposure to cadmium suggesting that sensory neurons may direct the specificity of the organismal stress response (FIG. 11). The AIY interneuron, on the other hand, appears to be required for gene induction upon exposure to both cadmium and heat, suggesting that this neuron may act as a node, or integrator of stress responses. We are currently in the process of confirming and extending these findings by using fluorescent reporters to confirm the cell non-autonomous nature of this regulation, and assaying whether such regulation extends to numerous cadmium-induced, including those induced in the ER (hsp-4), whether this regulation occurs both during development and through adulthood accounting for age-dependent changes in stress responsiveness, and which of the three major transcription factors are involved.

### Materials and Methods

C. elegans Strains Used

The following C. elegans strains were used: C. elegans Bristol wild type N2, C12C8.1p::GFP, and rrf-3 (pk1426)II. The rrf-3 (pk1426) loss of function mutation is a 6017 bp deletion (Simmer F. et al, 2002). AM597 is the strain that was constructed by crossing t rrf-3 (pk1426) with C12C8.1p::GFP.

### Crosses to Generate Specific Strain:

AM597 was obtained by crossing adult male rrf-3(pk1426) with C12C8.1p::GFP L4 hermaphrodites. F1 progeny was checked for the rrf-3/+ heterozygous genotype using PCR. F2 worms were checked for rrf-3/rrf-3 genotype using PCR. Heat shock and reporter expression was used to verify that the animals were homozygous for the C12C8.1p::GFP transgene.

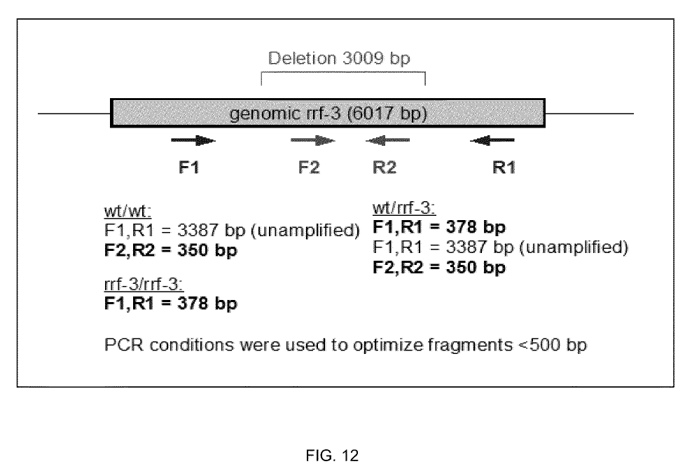

The PCR conditions for genotyping were optimized to amplify <500 bp products. Primers were designed flanking the rrf-3 deletion (F1/R1) or within the deletion (F2/R2) as depicted in FIG. 12.

### RNAi Screen

The bacteria OP50 (Brenner, 1974) was used for feeding C. elegans. The general methods for growing C. elegans were as described by Brenner (1974). As the quality of bacterial food and population densities greatly influences the activation of the HSR, great care was taken in the protocol to ensure optimized growing conditions for the worms throughout their development and prior to and during the RNAi experiment. AM597 worms were grown at low density: the F1 progeny of 5 L4 per plate were grown on standard NGM plates seeded with 200 to 300 uL of a stationary phase culture of OP50 grown in LB broth the day before were used. The bacterial culture was grown overnight and kept no more than 3 days in the fridge. Subsequently, 10 L4 progeny were picked onto RNAi plates seeded with 200 uL RNAi bacteria. Bacteria from the RNAi library were grown the day before in 3 mL LB containing ampicillin and tetracycline ([amp]=0.1 mg/mL, [tet]=0.4 mM), seeded onto the RNAi plates and allowed to grow at room temperature for 24 hours before plating the animals onto them. RNAi plates contained standard amounts of NGM ampicillin (100 ug/ml), tetracycline (12.5 ug/ml) and IPTG. (0.4 mM). 10-15 AM597 animals were transferred as L4 onto the RNAi plates and 24 hours later were subjected to heat shock treatment at 34° C. during 15 minutes. GFP fluorescence was monitored constantly, and animals were scored 2 h after heat shock.

RNAi against gcy-8 and ttx-3 were used as negative controls for RNAi induced knockdown of heat shock-dependent C12C8.1p::GFP expression in the AM597 animals. Animals fed with control HT115 were used as positive controls. Due to the imperfect pentrance of RNAi despite using a sensitized background and the resulting variability, we scored as hits, only those genes whose RNAi markedly reduced GFP expression consistently in at least 3 separate experiments in 50% or more of the animals. The underlying assumption in scoring the number of fluorescent animals as a measure of heat shock dependent C12C8.1p::GFP induction was that there would be a direct correlation between the number of animals expressing GFP and the proportion of hsp70 (C12C8.1) mRNA obtained from a population of animals. Animals were therefore classified into 2 categories.

+: as fluorescent as control;

−: less fluorescent than control or no fluorescence.

Since the number of worms fluorescent did not follow a Gaussian distribution, the non-parametric Wilcoxon test was used to analyze the data. To account for the variability between the experiments, the Wilcoxon pair-matched two-tailed test was used to compare the mean of control fluorescent animals with those that were as fluorescent as controls in each RNAi experiment.

While this invention has been particularly shown and described with references to preferred embodiments thereof, it will be understood by those skilled in the art that various changes in form and details may be made therein without departing from the scope of the invention encompassed by the appended claims.

1. A method of activating a heat shock response (HSR) in a first cell of a multicellular organism comprising increasing the activity of an HSR signaling factor,
wherein the HSR signaling factor mediates heat shock activation in the first cell,
wherein the HSR signaling factor is released from a second cell, and
wherein the second cell is a neuronal cell.

2. The method of claim 1 wherein the second cell does not directly innervate the first cell.

3. The method of claim 1 wherein the activity of the HSR signaling factor is increased by stimulating its release from the second cell.

4. The method of claim 1 wherein the activity of the HSR signaling factor is increased by agonizing a receptor of the HSR signaling factor.

5. The method of claim 1 wherein the HSR signaling factor is a ligand of the insulin like (IL)/insulin like growth factor (IGF) signaling pathway.

6. The method of claim 1 wherein the HSR signaling factor is a ligand of the transforming growth factor β (TGF-β) signaling pathway.

7. The method of claim 1 wherein the HSR signaling factor is a ligand of the steroid hormone pathway or is a neuropeptide.

8. (canceled)

9. The method of claim 1 wherein the activity of the HSR signaling factor is increased by administering a pharmacologic agent.

10. The method of claim 1 wherein the heat shock response is activated by increasing the expression of HSF-1 in the first cell.

11. The method of claim 1 wherein the first cell is a non-neuronal cell.

12. The method of claim 1 wherein the second cell is a thermosensory neuron.

13. The method of claim 1 wherein gene expression of a heat shock protein selected from the group consisting of the HSP60 family, the HSP70 family, the HSP90 family, the HSP27 family and the αB-crystallin family of proteins is increased in the first cell.

14. The method of claim 1 wherein the multicellular organism is a mammal.

15. The method of claim 14 wherein the mammal is a human.

16. A method of suppressing a heat shock response (HSR) in a first cell of a multicellular organism comprising inhibiting the activity of an HSR signaling factor,
wherein the HSR signaling factor mediates heat shock activation in the first cell,
wherein the HSR signaling factor is released from a second cell,
and wherein second cell is a neuronal cell.

17. The method of claim 16 wherein the second cell does not directly innervate the first cell.

18. The method of claim 16 wherein the activity of the HSR signaling factor is inhibited by inhibiting the release of the HSR signaling factor from the second cell.

19. The method of claim 16 wherein the activity of the HSR signaling factor is inhibited by antagonizing a receptor of the HSR signaling factor.

20-31. (canceled)

32. A method of treating a patient suffering from a condition associated with a dysfunction in the homeostasis of a protein in a first cell comprising stimulating the activity of an HSR signaling factor, wherein the HSR signaling factor is released from a second cell, wherein the HSR signaling factor mediates heat shock activation in the first cell and wherein the second cell is a neuronal cell.

33. The method of claim 32 wherein the second cell does not directly innervate the first cell.

34. The method of claim 32 wherein the activity of the HSR signaling factor is stimulated by administering a pharmacologic agent to the patient.

35. The method of claim 32 wherein the pharmacologic agent stimulates the release of a HSR signaling factor from the second cell or agonizes a receptor of the HSR signaling factor.

36. (canceled)

37. The method of claim 32 wherein HSR signaling factor is a ligand of the insulin like (IL)/insulin like growth factor (IGF) signaling pathway.

38. The method of claim 32 wherein the HSR signaling factor is a ligand of the transforming growth factor β (TGF-β) signaling pathway.

39. The method of claim 32 wherein the HSR signaling factor is a ligand of the steroid hormone pathway or is a neuropeptide like molecule.

40. (canceled)

41. The method of claim 32 wherein the condition associated with a dysfunction in protein homeostasis is selected from the group consisting of a loss of function disorder and a gain of function disorder.

42. (canceled)

43. The method of claim 41 wherein the gain of function disorder is a neurodegenerative disease.

44. The method of claim 43 wherein the neurodegenerative disease is selected from the group consisting of amyotrophic lateral sclerosis, Huntington's disease, dentatorubral atrophy, pallidoluysian atrophy, spino-cerebellar ataxia, Alzheimer's disease, senile systemic amyloidoses, familial amyloidotic neuropathy, and Parkinson's disease.

45. (canceled)

46. The method of claim 41 wherein the loss of function disorder is selected from the group consisting of cystic fibrosis and a lysosomal storage disease.

47. The method of claim 33 further comprising the administration of a compound that increases HSF-1 activity.

48. (canceled)

49. A method of treating a patient suffering from a condition associated with increased expression of a heat shock protein in a first cell comprising inhibiting the activity of an HSR signaling factor, wherein the HSR signaling factor is released from a second cell, wherein the HSR signaling factor mediates heat shock activation in a first cell and wherein the second cell is a neuronal cell.

50-63. (canceled)

64. A method of activating the HSR in a first cell of a multicellular organism comprising stimulating TGF-beta signaling activity of a second cell, wherein the second cell is a neuronal cell.

65. The method of claim 64 wherein the second cell does not directly innervate the first cell.

66. A method of decreasing the HSR in a multicellular response comprising inhibiting TGF-beta signaling activity of a second cell, wherein the second cell is a neuronal cell.

67. The method of claim 66 wherein the second cell does not directly innervate the first cell.

68. The method of claim 64 wherein the multicellular organism is a mammal and the TGF-beta signaling activity is mediated by the activity of a mammalian homologue of DBL-1.

69-71. (canceled)
